SEQUENCE LISTING (41 sequences)

SEQ ID NO: 1 - DNA, 399 bp, Artificial Sequence
  polynucleotide encoding a tau repeat domain
gcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacatgtctcgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttc

SEQ ID NO: 2 - DNA, 714 bp, Artificial Sequence
  Clover3 reporter
gtgagcaagggcgaggagctgttcaccggggtggtgcccatcctggtcgagctggacggcgacgtaaacggccacaagttcagcgtccgcggcgagggcgagggcgatgccaccaacggcaagctgaccctgaagttcatctgcaccaccggcaagctgcccgtgccctggcccaccctcgtgaccaccttcggctacggcgtggcctgcttcagccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgagctgaagggcatcgacttcaaggaggacggcaacatcctggggcacaagctggagtacaacttcaacagccactacgtctatatcacggccgacaagcagaagaactgcatcaaggctaacttcaagatccgccacaacgttgaggacggcagcgtgcagctcgccgaccactaccagcagaacacccccatcggcgacggccccgtgctgctgcccgacaaccactacctgagccatcagtccaagctgagcaaagaccccaacgagaagcgcgatcacatggtcctgctggagttcgtgaccgccgccgggattacacatggcatggacgagctgtacaag

SEQ ID NO: 3 - DNA, 714 bp, Artificial Sequence
  Cerulean3 reporter
gtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgagctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatccacggcaacgtctatatcaccgccgacaagcagaagaacggcatcaaggccaacttcggcctcaactgcaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaag

SEQ ID NO: 4 - DNA, 581 bp, Artificial Sequence
  CMV Promoter
atagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcc

SEQ ID NO: 5 - DNA, 36 bp, Artificial Sequence
  linker
ggttctgctggctccgctgctggatccggcgaattc

SEQ ID NO: 6 - DNA, 9735 bp, Artificial Sequence
  FM5 CMV 246-378aa P301S Tau mClover3
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgccgccaccatggcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacatgtctcgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttcggttctgctggctccgctgctggatccggcgaattcgtgagcaagggcgaggagctgttcaccggggtggtgcccatcctggtcgagctggacggcgacgtaaacggccacaagttcagcgtccgcggcgagggcgagggcgatgccaccaacggcaagctgaccctgaagttcatctgcaccaccggcaagctgcccgtgccctggcccaccctcgtgaccaccttcggctacggcgtggcctgcttcagccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgagctgaagggcatcgacttcaaggaggacggcaacatcctggggcacaagctggagtacaacttcaacagccactacgtctatatcacggccgacaagcagaagaactgcatcaaggctaacttcaagatccgccacaacgttgaggacggcagcgtgcagctcgccgaccactaccagcagaacacccccatcggcgacggccccgtgctgctgcccgacaaccactacctgagccatcagtccaagctgagcaaagaccccaacgagaagcgcgatcacatggtcctgctggagttcgtgaccgccgccgggattacacatggcatggacgagctgtacaagtaaggcgcgccgggatcgtacaagtaaagcggccgcgactctagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 7 - DNA, 9735 bp, Artificial Sequence
  FM5 CMV 246-378aa P301S Tau mCerulean3
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgccgccaccatggcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacatgtctcgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttcggttctgctggctccgctgctggatccggcgaattcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgagctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatccacggcaacgtctatatcaccgccgacaagcagaagaacggcatcaaggccaacttcggcctcaactgcaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaaggcgcgccgggatcgtacaagtaaagcggccgcgactctagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 8 - protein, 5 aa, Artificial Sequence
  GGGGS Linker (n=1)
GGGGS

SEQ ID NO: 9 - protein, 10 aa, Artificial Sequence
  GGGGS Linker (n=2)
GGGGSGGGGS

SEQ ID NO: 10 - protein, 15 aa, Artificial Sequence
  GGGGS Linker (n=3)
GGGGSGGGGSGGGGS

SEQ ID NO: 11 - protein, 8 aa, Artificial Sequence
  Linker
GGGGGGGG

SEQ ID NO: 12 - protein, 6 aa, Artificial Sequence
  Linker
GGGGGG

SEQ ID NO: 13 - protein, 15 aa, Artificial Sequence
  EAAAK Linker (n=3)
EAAAKEAAAKEAAAK

SEQ ID NO: 14 - protein, 5 aa, Artificial Sequence
  EAAAK Linker (n=1)
EAAAK

SEQ ID NO: 15 - protein, 10 aa, Artificial Sequence
  Linker (n=2)
EAAAKEAAAK

SEQ ID NO: 16 - protein, 46 aa, Artificial Sequence
  Linker
AEAAAKEAAAKEAAAKEAAAKALEAEAAAKEAAAKEAAAKEAAAKA

SEQ ID NO: 17 - protein, 5 aa, Artificial Sequence
  Linker
PAPAP

SEQ ID NO: 18 - protein, 12 aa, Artificial Sequence
  Linker
AEAAAKEAAAKA

SEQ ID NO: 19 - protein, 20 aa, Artificial Sequence
  GGGGS Linker (n=4)
GGGGSGGGGSGGGGSGGGGS

SEQ ID NO: 20 - protein, 10 aa, Artificial Sequence
  Ala Pro Linker (n=5)
APAPAPAPAP

SEQ ID NO: 21 - protein, 12 aa, Artificial Sequence
  Ala Pro Linker (n=6)
APAPAPAPAPAP

SEQ ID NO: 22 - protein, 14 aa, Artificial Sequence
  Ala Pro Linker (n=7)
APAPAPAPAPAPAP

SEQ ID NO: 23 - protein, 16 aa, Artificial Sequence
  Ala Pro Linker (n=8)
APAPAPAPAPAPAPAP

SEQ ID NO: 24 - protein, 18 aa, Artificial Sequence
  Ala Pro Linker (n=9)
APAPAPAPAPAPAPAPAP

SEQ ID NO: 25 - protein, 20 aa, Artificial Sequence
  Ala Pro Linker (n=10)
APAPAPAPAPAPAPAPAPAP

SEQ ID NO: 26 - protein, 22 aa, Artificial Sequence
  Ala Pro Linker (n=11)
APAPAPAPAPAPAPAPAPAPAP

SEQ ID NO: 27 - protein, 24 aa, Artificial Sequence
  Ala Pro Linker (n=12)
APAPAPAPAPAPAPAPAPAPAPAP

SEQ ID NO: 28 - protein, 26 aa, Artificial Sequence
  Ala Pro Linker (n=13)
APAPAPAPAPAPAPAPAPAPAPAPAP

SEQ ID NO: 29 - protein, 28 aa, Artificial Sequence
  Ala Pro Linker (n=14)
APAPAPAPAPAPAPAPAPAPAPAPAPAP

SEQ ID NO: 30 - protein, 30 aa, Artificial Sequence
  Ala Pro Linker (n=15)
APAPAPAPAPAPAPAPAPAPAPAPAPAPAP

SEQ ID NO: 31 - protein, 32 aa, Artificial Sequence
  Ala Pro Linker (n=16)
APAPAPAPAPAPAPAPAPAPAPAPAPAPAPAP

SEQ ID NO: 32 - protein, 34 aa, Artificial Sequence
  Ala Pro Linker (n=17)
APAPAPAPAPAPAPAPAPAPAPAPAPAPAPAPAP

SEQ ID NO: 33 - protein, 17 aa, Artificial Sequence
  Linker
VSQTSKLTRAETVFPDV

SEQ ID NO: 34 - protein, 6 aa, Artificial Sequence
  Linker
PLGLWA

SEQ ID NO: 35 - protein, 16 aa, Artificial Sequence
  Linker
RVLAEAEDVVCCSMSY

SEQ ID NO: 36 - protein, 8 aa, Artificial Sequence
  Linker
GGIEGRGS

SEQ ID NO: 37 - protein, 10 aa, Artificial Sequence
  Linker
TRHRQPRGWE

SEQ ID NO: 38 - protein, 10 aa, Artificial Sequence
  Linker
AGNRVRRSVG

SEQ ID NO: 39 - protein, 9 aa, Artificial Sequence
  Linker
RRRRRRRRR

SEQ ID NO: 40 - protein, 4 aa, Artificial Sequence
  Linker
GFLG

SEQ ID NO: 41 - protein, 12 aa, Artificial Sequence
  Linker
GSAGSAAGSGEF

(19) United States
(12) Patent Application Publication
(10) Pub. No.: US 2024/0043483 A1
(43) Pub. Date: Feb. 8, 2024

(54) TAU BIOSENSOR CELL LINES

(71) Applicant: THE BOARD OF REGENTS OF THE UNIVERSITY OF TEXAS SYSTEM, Austin, TX (US)

(72) Inventors: Marc DIAMOND, Dallas, TX (US); Victor MANON, Dallas, TX (US); Jaime VAQUER-ALICEA, Dallas, TX (US); Brian HITT, Dallas, TX (US)

(73) Assignee: THE BOARD OF REGENTS OF THE UNIVERSITY OF TEXAS SYSTEM, Austin, TX (US)

(21) Appl. No.: 18/266,712

(22) PCT Filed: Dec. 21, 2021
(86) PCT No.: PCT/US2021/064590

§ 371 Date: Jun. 12, 2023

Related U.S. Application Data
(60) Provisional application No. 63/128,370, filed on Dec. 21, 2020.

(51) Int. Cl. C07K 14/47; C12N 15/63; G01N 33/68; G01N 33/50

(52) U.S. Cl. CPC C07K 14/4711; C12N 15/63; G01N 33/6896; G01N 33/5058

(57) ABSTRACT

The present disclosure provides biosensor cells and methods of use thereof. The disclosure provides, for example, methods of measuring a titer of or of detecting a seed tau protein in a sample, methods of detecting Alzheimer's disease (AD), or a neurodegenerative tauopathy disease or condition linked to tau protein aggregation, and methods for the identification of putative tau protein aggregation inhibitors or modulators.

### CROSS-REFERENCE TO RELATED APPLICATIONS

This application claims the benefit of priority under 35 U.S.C. § 119(e) to U.S. Ser. No. 63/128,370 filed Dec. 21, 2020, which is herein incorporated by reference in its entirety.

### INCORPORATION OF SEQUENCE LISTING

The material in the accompanying sequence listing is hereby incorporated by reference into this application. The accompanying sequence listing text file, name 426871-000126-Sequence_Listing_ST25, was created on Dec. 21, 2021, and is 41 kb. The file can be accessed using Microsoft Word on a computer that uses Windows OS.

### FIELD OF THE INVENTION

The present disclosure relates to methods for the measurement or detection of pathological tau protein conformers (monomers, assemblies), the detection of tau protein aggregation-related diseases or disorders, and the identification of tau protein aggregation inhibitors or modulators.

### BACKGROUND

Amyloid-forming proteins having a “seeding” activity and capable of prion-like self-replication, such as tau protein, are responsible for tauopathies such as Alzheimer's disease (AD). In AD, tau progressively accumulates via the formation of aggregate “seeds” in a single neuron or group of neurons that exit and then gain entry to neighboring or synaptically connected cells, indicating that seed formation is the earliest detectable pathological event. A seed can range in size from a protein monomer to a multimeric assembly.

Reliable detection of seeding activity in peripheral fluids such as CSF or blood from living subjects has not yet been established. There is an unmet need for a highly sensitive biosensors that can be reliably used to detect tau seeding in CSF, and for methods of detecting the smallest amount of tau protein seed possible in a sample to ensure the earliest possible diagnosis of the disease.

### SUMMARY

Provided herein are polynucleotides, expression cassettes, vectors, cells comprising vectors, and methods of uses thereof. Specifically, provided herein are methods of measuring a titer of or of detecting a seed tau protein in a sample, methods of detecting Alzheimer's disease (AD), or a neurodegenerative tauopathy disease or condition linked to tau protein aggregation, methods for the identification of putative tau protein aggregation inhibitor, and methods of detecting attomolar levels of seed tau protein.

An embodiment provides a polynucleotide comprising: a polynucleotide encoding a tau repeat domain comprising SEQ ID NO:1; and a polynucleotide encoding a reporter.

A polynucleotide can further comprise a polynucleotide encoding a promoter; and a polynucleotide encoding a linker. The polynucleotide encoding a reporter can comprise SEQ ID NO:2 or SEQ ID NO:3. The polynucleotide encoding a promoter can comprise SEQ ID NO:4. The polynucleotide encoding a linker can comprise SEQ ID NO:5.

Another embodiment provides a vector comprising an expression cassette comprising a polynucleotide encoding a tau repeat domain comprising SEQ ID NO:1 and a reporter.

The vector can comprise SEQ ID NO:6 or 7.

An embodiment provides a cell comprising: (i) a first vector comprising a polynucleotide encoding a tau repeat domain and a first reporter, and a second vector comprising a polynucleotide encoding a tau repeat domain and a second reporter; or (ii) a vector comprising a first polynucleotide encoding a tau repeat domain and a first reporter, and a second polynucleotide encoding a tau repeat domain and a second reporter. The first reporter can comprise SEQ ID NO:2 or SEQ ID NO:3. The first polynucleotide can comprise SEQ ID NO:1 and SEQ ID NO:2. The second polynucleotide can comprise SEQ ID NO:1 and SEQ ID NO:3. The first polynucleotide can comprise SEQ ID NO:6, and the second polynucleotide can comprise SEQ ID NO:7. The cell can express Tau RD(P301S).

An embodiment provides a method of measuring a titer of or of detecting a seed tau protein in a sample comprising: contacting the sample with a population of the cells described herein; performing a seeding assay; and detecting tau protein aggregates, thereby measuring a titer of or of detecting seed tau protein in the sample.

Another embodiment provides a method of detecting Alzheimer's disease (AD), or a neurodegenerative tauopathy disease or condition linked to tau protein aggregation in a subject comprising: contacting a sample with a population of the cells described herein; performing a seeding assay; and detecting tau protein aggregates, thereby detecting AD or neurodegenerative tauopathy disease or condition in a subject.

The sample can be comprised of recombinant protein, a biological fluid, a tissue sample, a cerebrospinal fluid, a brain homogenate, or an aggregated material amplified in vitro therefrom. Tau protein present in the sample can be immunoprecipitated prior to contacting the sample with the cell described herein. The method can detect about as low as 10 pg/ml of tau protein in the sample.

An embodiment provides a method of identifying a tau protein aggregation inhibitor comprising: contacting a population of the cells described herein with a putative tau protein aggregation inhibitor; performing a seeding assay; detecting tau protein aggregates, and identifying a tau protein aggregation inhibitor, wherein a tau protein aggregation inhibitor interacts with tau protein.

Detecting tau protein aggregates can indicate that the putative tau protein aggregation inhibitor does not inhibit tau protein aggregation. A lack of detection of tau protein aggregates can indicate that the putative tau protein aggregation inhibitor inhibits tau protein aggregation.

Another embodiment provides a method of detecting attomolar levels of a seed tau protein in a sample comprising: contacting the sample with the cell described herein; performing a seeding assay; and detecting tau protein aggregates, thereby detecting seed tau protein present at attomolar levels in the sample.

Another embodiment provides a method of identifying tau protein aggregation regulator or modulator comprising contacting a population of the cells described herein with a putative tau protein aggregation regulator or modulator; performing a seeding assay; and detecting change in tau protein aggregation in the cell, thereby identifying putative tau protein aggregation regulator or modulator.

The tau protein aggregation regulator or modulator can be a small molecule, a nucleic acid, a protein or a metabolic factor. Detecting more tau protein aggregates in the presence of a putative tau protein aggregation regulator or modulator can indicate that the putative tau protein aggregation regulator or modulator induces or promotes tau protein aggregation. Detecting less tau protein aggregates in the presence of a putative tau protein aggregation regulator or modulator can indicate that the putative tau protein aggregation regulator or modulator inhibits or prevents tau protein aggregation.

### BRIEF DESCRIPTION OF THE DRAWINGS

The features, objects and advantages other than those set forth above will become more readily apparent when consideration is given to the detailed description below. Such detailed description makes reference to the following drawings, wherein:

FIG. 1 shows the map of a vector including a P301S tau protein and a mClover3 reporter.

FIG. 2 shows the map of a vector including a P301S tau protein and a mCerulean3 reporter.

FIG. 3 shows expression levels of tau-fusion proteins in v2H cells as compared to v1 biosensors cells. FIG. 3A shows an immunoblot against Tau. FIG. 3B shows an immunoblot against YFP. FIG. 3C is a fluorescence micrograph of v1 cells examined under the YFP channel. FIG. 3D is a fluorescence micrograph of v2H cells examined under the YFP channel.

FIG. 4 shows improved sensitivity of P301S v2H biosensor cells over P301S v1 cells. FIG. 4A is a graph illustrating FRET positivity from treatment with successive dilutions of synthetic tau fibrils with Lipofectamine 2000. FIG. 4B is a graph illustrating expansions of the low end of the curves in FIG. 4A.

FIG. 5 shows improved sensitivity of P301S v2H biosensor cells over P301S v1 cells. FIG. 5A is a graph illustrating FRET positivity from treatment with successive dilutions of synthetic tau fibrils without transfection reagent (log scale). FIG. 5B is a graph illustrating expansions of the low end of the curves in FIG. 5A.

FIG. 6 shows tau seeding activity from biological sources. FIG. 6A shows dose response curves of P301S v2 cells with protein from PS19 transgenic, wild-type, and tau knock-out mouse brain. FIG. 6B is a dose response curves of P301S v2 cells using protein from frontal lobe tissue of 5 AD cases. FIG. 6C is an expansion of the low end of FIG. 6A. FIG. 6D is an expansion of the low end of FIG. 6B.

FIG. 7 illustrates that tau seeds can efficiently be purified from CSF. FIG. 7A is a graft bar illustrating FRET positivity resulting from IP followed by seeding assay of 10 ng of protein from frontal cortex of case AD1 was spiked into control CSF or PBS with different IP volume. FIG. 7B is a graft bar illustrating FRET positivity of 1 ml aliquots of control CSF spiked with a serial dilution of protein from brain AD1. FIG. 7C is a graph bar illustrating FRET positivity of 1 ml aliquots of control CSF spiked with a serial dilution of recombinant tau fibrils.

### DETAILED DESCRIPTION

### Overview

There is increasing evidence that the accumulation and spread of protein aggregates in neurodegenerative diseases, such as Alzheimer's Disease and Parkinson's Disease occurs via prion or prion-like mechanisms. According to this model, a natively folded protein undergoes a conformational change and becomes capable of forming pathogenic aggregates. These aggregates then act as templates for self-replication as they spread from cell to cell. Ultimately, this process leads to cellular dysfunction and neurodegeneration.

Intracellular aggregates of the microtubule-associated protein tau define Alzheimer's disease (AD) and related neurodegenerative tauopathies. In AD, tau progressively accumulates in defined patterns that involve brain networks. The formation of aggregate “seeds” in a single neuron or group of neurons can exit and then gain entry to neighboring or synaptically connected cells. The seeds then serve as templates for amplification of specific pathological tau assemblies. Accordingly, assays to measure the titer of tau aggregates in human brain or samples prepared in vitro can be useful for diagnosis and drug discovery.

Provided herein are polynucleotides encoding a tau repeat domain and a reporter, which, when expressed in host cell lines, enable the detection of tau protein. These biosensor cell lines, constitute tools for clinical diagnosis and for drug discovery.

Indeed, using the biosensor cells, the methods described herein allow for the rapid detection of tau protein at the attomolar level, and therefore can also be used to assist in the discovery of novel drugs that can bind pathogenic seed tau protein, or that can interfere with its replication in cells.

Therefore, the methods described herein allow for the detection of seed tau protein, the detection of seed tau protein-related diseases or disorders, and the identification of seed tau protein aggregation inhibitors.

Polynucleotides

An embodiment provides a polynucleotide encoding a tau repeat domain and a reporter.

Polynucleotides

Polynucleotides refer to nucleic acid molecules comprising deoxyribonucleic acid (DNA) or ribonucleic acid (RNA). Nucleic acid molecules include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugated, oligonucleotides or combinations thereof. Polynucleotides can be present as a single-stranded or double-stranded and linear or covalently circularly closed molecule.

Polynucleotides can be obtained from nucleic acid molecules present in, for example, a mammalian cell. Polynucleotides can also be synthesized in the laboratory, for example, using an automatic synthesizer. An amplification method such as PCR can be used to amplify polynucleotides from either genomic DNA or cDNA.

Polynucleotides can be isolated. An isolated polynucleotide can be a naturally occurring polynucleotide that is not immediately contiguous with one or both of the 5′ and 3′ flanking genomic sequences that it is naturally associated with. An isolated polynucleotide can be, for example, a recombinant DNA molecule of any length, provided that the nucleic acid molecules naturally found immediately flanking the recombinant DNA molecule in a naturally occurring genome is removed or absent. Isolated polynucleotides also include non-naturally occurring nucleic acid molecules. Polynucleotides can encode full-length polypeptides, polypeptide fragments, and variant or fusion polypeptides. “Isolated polynucleotides” can be (i) amplified in vitro, for example via polymerase chain reaction (PCR), (ii) produced recombinantly by cloning, (iii) purified, for example, by cleavage and separation by gel electrophoresis, (iv) synthesized, for example, by chemical synthesis, or (vi) extracted from a sample.

A polynucleotide can comprise, for example, a gene, open reading frame, non-coding region, or regulatory element. A gene is any polynucleotide that encodes a polypeptide, protein, or fragment thereof, optionally including one or more regulatory elements preceding (5′ non-coding sequences) and following (3′ non-coding sequences) the coding sequence. In one embodiment, a gene does not include regulatory elements preceding and following the coding sequence. A native or wild-type gene refers to a gene as found in nature, optionally with its own regulatory elements preceding and following the coding sequence. A chimeric or recombinant gene refers to any gene that is not a native or wild-type gene, optionally comprising regulatory elements preceding and following the coding sequence, wherein the coding sequences and/or the regulatory elements, in whole or in part, are not found together in nature. Thus, a chimeric gene or recombinant gene comprise regulatory elements and coding sequences that are derived from different sources, or regulatory elements and coding sequences that are derived from the same source but arranged differently than is found in nature. A gene can encompass full-length gene sequences (e.g., as found in nature and/or a gene sequence encoding a full-length polypeptide or protein) and can also encompass partial gene sequences (e.g., a fragment of the gene sequence found in nature and/or a gene sequence encoding a protein or fragment of a polypeptide or protein). A gene can include modified gene sequences (e.g., modified as compared to the sequence found in nature). Thus, a gene is not limited to the natural or full-length gene sequence found in nature.

Polynucleotides can be purified free of other components, such as proteins, lipids and other polynucleotides. For example, the polynucleotide can be 50%, 75%, 90%, 95%, 96%, 97%, 98%, 99%, or 100% purified. A polynucleotide existing among hundreds to millions of other polynucleotides within, for example, cDNA or genomic libraries, or gel slices containing a genomic DNA restriction digest are not to be considered a purified polynucleotide. Polynucleotides can encode the polypeptides described herein (e.g., any tau polypeptide, seed tau polypeptide, fragments or variants thereof suitable for the use described herein).

Degenerate polynucleotide sequences encoding polypeptides described herein, as well as homologous nucleotide sequences that are at least about 80, or about 85, 90, 91, 92, 93, 94, 95, 96, 97, 98, or 99% identical to polynucleotides described herein and the complements thereof are also polynucleotides. Degenerate nucleotide sequences are polynucleotides that encode a polypeptide described herein or fragments thereof but differ in nucleic acid sequence from the wild-type polynucleotide sequence, due to the degeneracy of the genetic code. Complementary DNA (cDNA) molecules, species homologs, and variants of polynucleotides that encode biologically functional polypeptides also are polynucleotides.

Polynucleotides can comprise coding sequences for naturally occurring polypeptides or can encode altered sequences that do not occur in nature.

Unless otherwise indicated, the term polynucleotide or gene includes reference to the specified sequence as well as the complementary sequence thereof.

The expression products of genes or polynucleotides are often proteins, or polypeptides, but in non-protein coding genes such as rRNA genes or tRNA genes, the product is a functional RNA. The process of gene expression is used by all known life forms, i.e., eukaryotes (including multicellular organisms), prokaryotes (bacteria and archaea), and viruses, to generate the macromolecular machinery for life. Several steps in the gene expression process can be modulated, including the transcription, up-regulation, RNA splicing, translation, and post-translational modification of a protein.

Tau Repeat Domain

Polynucleotides can encode a tau repeat domain. Tau is a natively unstructured protein expressed as 6 isoforms in the adult human brain that result from alternative splicing of the MAPT gene. Tau is mainly known for its ability to stabilize microtubules within axons of neurons. Tau isoforms are composed of either 3 or 4 microtubule-binding repeats (MTBRs; 3R or 4R), which mediate binding of tau to microtubules. Aberrant misfolding of tau leads to fibrillization and the formation of paired helical filaments with all 6 tau isoforms that constitute neurofibrillary tangles (NFTs).

Misfolded tau, or “tau seeds” are capable of initiating aggregation of various forms of tau. As used herein, the term “tau seed” refers to a tau aggregate—or seed—that is a misfolded tau protein or fragment thereof, capable of recruiting normal, soluble tau into a fibrillar conformation. Tau seeds can spread, transmitting the aggregated tau from cell to cell via prion-like mechanisms. Upon uptake and processing, the misfolded seed tau seed can initiate templated fibrilization and recruit native tau monomer by direct protein-protein interactions between a pathological tau seed and naive cellular tau, to form new pathologic fibril in the recipient cell. The conversion of a protein from a monomer to a large, ordered multimer can occur by several mechanisms, but the first step likely involves the formation of a seed. A seed is potentially transitory, arising from an equilibrium between two states: one relatively aggregation-resistant, and another that is short-lived. A seed could be a single molecule, or several. Based on extrapolation from kinetic aggregation studies, it is likely that a critical seed for tau and polyglutamine peptide amyloid formation is a single molecule or a tau multimer. Therefore, the term “seed” is used to refer to the structure that serves as a template for homotypic fibril growth and can range in size from a protein monomer to a multimeric assembly. For example, a seed can refer to any misfolded protein capable of initiating aggregation of various forms of tau, and can therefore comprise 1, 2, 3, 4, 5, 10, 20, 30, 40, 50 or more monomers or 50, 40, 30, 20, 10, 5, 4, 3, 2 or less monomers.

As used herein a “tau repeat domain” refers to a domain or portion of a tau protein or fragment capable of forming self-replicating assemblies (e.g., tau protein or aggregates thereof capable of inducing protein-protein interaction and therefore further tau aggregates, and capable of transmission from one cell to the other). A “tau repeat domain” corresponds to a portion of a tau protein that can interact with another tau protein to form protein-protein interactions, and thus generate fibrils. A Tau repeat domain comprises, for example, three or four 31-32-residue imperfect repeats that form the core of tau filaments and is capable of self-assembling into filaments in vitro. Therefore, a “tau repeat domain” can be used to refer to a microtubule-binding repeat domain of a tau protein described herein, for use in, for example, the generation of biosensor cells. A Tau repeat domain can, for example, comprise SEQ ID NO:1.

In other embodiments, a tau repeat domain comprises one or more 31-32-residue imperfect repeats that form the core of tau filaments and is capable of self-assembling into filaments in vitro. For example, a tau repeat domain can comprise 1, 2, 3, 4, 5, or more 31-32-residue imperfect repeats. In some embodiments, a tau repeat domain can be about 25, 26, 27, 28, 29, 30, 31, 32, 33, 34, 35, 36, 37, 38, 39 or more amino acids in length. In some embodiments, a tau repeat domain can be about 39, 38, 37, 36, 35, 34, 33, 32, 31, 30, 29, 28, 27, 26, 25, or less amino acids in length.

For example, a seed tau protein can refer to a tau protein fragment that has a misfolded conformation, comprising a disease-associated mutation, which can confer a tau protein fragment the ability to form self-replicating assemblies. Seed tau protein can occur naturally and can for example be derived from the diseased brain (such as the brain of a diagnosed patient, or the brain of an animal model). Non-naturally occurring seed tau protein, such as synthetic tau preformed fibrils (PFFs) can act as seeds in a templated fibrillization reaction in which misfolded tau recruits and corrupts normal, soluble tau into a fibrillar conformation for generate tau aggregates.

There are two classes of tau gene mutations: missense mutations which alter the microtubule binding properties of tau protein and mutations that alter the splicing of exon 10 to produce an increase in tau mRNA with exon 10 inserts (comprising MTBRs, or tau repeat domain). Missense mutations are located in or around the microtubule binding domains and act via decreasing microtubule assembly, leading to filament destabilization and an increase in cytosolic tau. All tau gene mutations producing splicing defects increase the levels of four repeat tau isoforms that accumulate as insoluble aggregates in the brain.

Non limiting examples of disease-associated mutations include P301S. A polypeptide is a polymer of two or more amino acids covalently linked by amide bonds, and which can be encoded by a polynucleotide. A polypeptide can be post-translationally modified. A purified polypeptide is a polypeptide preparation that is substantially free of cellular material, other types of polypeptides, chemical precursors, chemicals used in synthesis of the polypeptide, or combinations thereof. A polypeptide preparation that is substantially free of cellular material, culture medium, chemical precursors, chemicals used in synthesis of the polypeptide, etc., has less than about 30%, 20%, 10%, 5%, 1% or more of other polypeptides, culture medium, chemical precursors, and/or other chemicals used in synthesis. Therefore, a purified polypeptide is about 70%, 80%, 90%, 95%, 99% or more pure. A purified polypeptide does not include unpurified or semi-purified cell extracts or mixtures of polypeptides that are less than 70% pure.

The term “polypeptides” can refer to one or more of one type of polypeptide (a set of polypeptides). “Polypeptides” can also refer to mixtures of two or more different types of polypeptides (a mixture of polypeptides). The terms “polypeptides” or “polypeptide” can each also mean “one or more polypeptides.”

As used herein, the term “polypeptide of interest” or “polypeptides of interest”, “protein of interest”, “proteins of interest” includes any or a plurality of any of the tau repeat domain, tau polypeptides, seed tau polypeptide, or other polypeptides (including fragment polypeptides) described herein. For example, a polypeptide of interest can be a seed tau protein.

A mutated protein or polypeptide comprises at least one deleted, inserted, and/or substituted amino acid, which can be accomplished via mutagenesis of polynucleotides encoding these amino acids. Mutagenesis includes well-known methods in the art, and includes, for example, site-directed mutagenesis by means of PCR or via oligonucleotide-mediated mutagenesis as described in Sambrook et al., Molecular Cloning—A Laboratory Manual, 2nd ed., Vol. 1-3 (1989).

The terms “sequence identity” or “percent identity” are used interchangeably herein. To determine the percent identity of two polypeptide molecules or two polynucleotide sequences, the sequences are aligned for optimal comparison purposes (e.g., gaps can be introduced in the sequence of a first polypeptide or polynucleotide for optimal alignment with a second polypeptide or polynucleotide sequence). The amino acids or nucleotides at corresponding amino acid or nucleotide positions are then compared. When a position in the first sequence is occupied by the same amino acid or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). In some embodiments the length of a reference sequence (e.g., SEQ ID NO:1, 6, or 7) aligned for comparison purposes is at least 80% of the length of the comparison sequence, and in some embodiments is at least 90% or 100%. In an embodiment, the two sequences are the same length.

Ranges of desired degrees of sequence identity are approximately 80% to 100% and integer values in between. Percent identities between a disclosed sequence and a claimed sequence can be at least 80%, at least 83%, at least 85%, at least 90%, at least 95%, at least 98%, at least 99%, at least 99.5%, or at least 99.9%. In general, an exact match indicates 100% identity over the length of the reference sequence (e.g., SEQ ID NO:1, 6, or 7).

Polypeptides and polynucleotides that are sufficiently similar to polypeptides and polynucleotides described herein can be used herein. Polypeptides and polynucleotides that are about 90, 91, 92, 93, 94 95, 96, 97, 98, 99 99.5% or more identical to polypeptides and polynucleotides described herein can also be used herein.

For example, a polynucleotide can have 80% 85%, 90%, 91%, 92%, 93%, 94%, 95%, 96%, 97%, 98%, 99% or more identity to SEQ ID NO:1, 6, or 7.

Polypeptides and polynucleotides that are sufficiently similar to polypeptides and polynucleotides described herein (e.g., seed tau protein or polypeptide fragment thereof) can be used herein. Polypeptides and polynucleotides that are about 85, 90, 91, 92, 93, 94 95, 96, 97, 98, 99 99.5% or more identical to polypeptides and polynucleotides described herein (e.g., seed tau protein fragment and variants thereof) can also be used herein.

In an embodiment, a polynucleotide can encode a tau repeat domain comprising SEQ ID NO:1.

Polynucleotides encoding a tau repeat domain can be operably linked to additional polynucleotides. For example, polynucleotides encoding tau repeat domains can be operably linked to a reporter. In an embodiment, polynucleotides can encode a reporter.

Reporter

As used herein a “reporter” refers to a molecule such as a polypeptide that can be detected using a method adapted to the detection of said reporter, to efficiently report the presence or absence of seed tau protein in a sample. For example, a reporter described herein can refer to a fluorescent protein, which can be detected using any technique capable of detecting fluorescence, such as fluorescent microscopy, flow cytometry, FRET, BRET, and the like. In other examples, a reporter can refer to a bioluminescent protein.

Fluorescent proteins are proteins characterized by their ability to absorb light at a certain wavelength (excitation), and to subsequently emit of secondary fluorescence at a longer wavelength (emission), which can be detected. The excitation and emission wavelengths are often separated from each other by tens to hundreds of nanometers. In an embodiment, the fluorescent proteins can be the members of a FRET pair.

Fluorescence resonance energy transfer or FRET can be used to determine if two fluorescent proteins are within a certain distance of each other. By using a mechanism relying on energy transfer between two light-sensitive fluorescent proteins, the interaction, or lack thereof, between two molecules can be detected: therefore, allowing the extremely sensitive detection of small changes in the distance between two molecules. The fundamental mechanism of FRET involves a donor fluorescent protein in an excited electronic state, which can transfer its excitation energy to a nearby acceptor fluorescent protein through a non-radiative long-range dipole-dipole interaction. The efficiency of this energy transfer being inversely proportional to the sixth power of the distance between donor and acceptor fluorescent proteins.

In the presence of a suitable acceptor, a donor fluorescent protein can transfer excited state energy directly to the acceptor without emitting a photon. The resulting fluorescence sensitized emission has characteristics similar to the emission spectrum of an acceptor.

A FRET proximity detection protein pair can comprise a donor fluorescent protein and an acceptor fluorescent protein having compatible excitation and emission wavelength, to allow the detection of an energy transfer.

Non-limiting examples of FRET proximity detection protein pairs include mClover3/mCerulean3, mClover3/mRuby3, EBFP2/mEGFP, ECFP/EYFP, Cerulean/Venus, MiCy/mKO, CyPet/YPet, EGFP/mCherry, Venus/mCherry, Venus/tdTomato, and Venus/mPlum.

In another embodiment, any proximity detection system for proteins, including fluorescence complementation, bioluminescence resonance energy transfer, split luciferase assay, and Split-APEX2 can be used.

Non-limiting examples of fluorescent protein reporters that can be used include green fluorescent protein (GFP), cyan fluorescent protein (CFP), yellow fluorescent protein (YFP), Ruby, Cherry, and mEOS.

In an embodiment, a FRET proximity detection protein pair can include mClover3 and mCerulean3. In an embodiment, a polynucleotide encoding a reporter can comprise SEQ ID NO: 2 or SEQ ID NO:3.

For example, a polynucleotide can encode a tau repeat domain comprising SEQ ID NO:1 and a polynucleotide encoding a reporter comprising SEQ ID NO:2; or the polynucleotide can encode a tau repeat domain comprising SEQ ID NO:1, and a reporter comprising SEQ ID NO:3.

Polynucleotides encoding a tau repeat domain and a reporter can be operably linked to additional regulatory elements necessary for the incorporation of the polynucleotide into expression cassette, or for their expression into host cells. For example, polynucleotides encoding tau repeat domain and a reporter can be operably linked to a promoter. In an embodiment, polynucleotides described herein can further encode a promoter.

Promoter

A promoter is a nucleotide sequence that is capable of controlling the expression of a coding sequence or gene. Promoters are generally located 5′ of the sequence that they regulate. Promoters can be derived in their entirety from a native gene or be composed of different elements derived from promoters found in nature, and/or comprise synthetic nucleotide segments. Those skilled in the art will readily ascertain that different promoters can regulate expression of a coding sequence or gene in response to a particular stimulus, e.g., in a cell- or tissue-specific manner, in response to different environmental or physiological conditions, or in response to specific compounds. Promoters are typically classified into two classes: inducible and constitutive. A constitutive promoter refers to a promoter that allows for continual transcription of the coding sequence or gene under its control.

An inducible promoter refers to a promoter that initiates increased levels of transcription of the coding sequence or gene under its control in response to a stimulus or an exogenous environmental condition. If inducible, there are inducer polynucleotides present therein that mediate regulation of expression so that the associated polynucleotide is transcribed only when an inducer molecule is present. A directly inducible promoter refers to a regulatory region, wherein the regulatory region is operably linked to a gene encoding a protein or polypeptide, where, in the presence of an inducer of the regulatory region, the protein or polypeptide is expressed. An indirectly inducible promoter refers to a regulatory system comprising two or more regulatory regions, for example, a first regulatory region that is operably linked to a first gene encoding a first protein, polypeptide, or factor, e.g., a transcriptional regulator, which is capable of regulating a second regulatory region that is operably linked to a second gene, the second regulatory region may be activated or repressed, thereby activating or repressing expression of the second gene. Both a directly inducible promoter and an indirectly inducible promoter are encompassed by inducible promoter.

A promoter can be any polynucleotide that shows transcriptional activity in a chosen host cell. A promoter can be naturally occurring, can be composed of portions of various naturally occurring promoters, or may be partially or totally synthetic. Guidance for the design of promoters is derived from studies of promoter structure, such as that of Harley and Reynolds, Nucleic Acids Res., 15, 2343-61 (1987). In addition, the location of the promoter relative to the transcription start can be optimized. Many suitable promoters for use in mammalian cells are well known in the art, as are polynucleotides that enhance expression of an associated expressible polynucleotide. Non-limiting examples of constitutive promoters that can be used to in expression cassettes can include, for example, cytomegalovirus (CMV) promoter and Rous sarcoma virus promoter, which allow for unregulated expression in mammalian cells.

In an embodiment, a polynucleotide encoding a promoter can comprise SEQ ID NO:4.

For example, a polynucleotide can comprise a polynucleotide encoding a polynucleotide encoding a promoter comprising SEQ ID NO:4, a tau repeat domain comprising SEQ ID NO:1, and a polynucleotide encoding a reporter comprising SEQ ID NO:2; or a polynucleotide can comprise a polynucleotide encoding a promoter comprising SEQ ID NO:4, a polynucleotide encoding a tau repeat domain comprising SEQ ID NO:1, and a polynucleotide encoding a reporter comprising SEQ ID NO:3.

Polynucleotides can be operably linked with one another through a short polynucleotide sequence encoding a linker. For example, a polynucleotide encoding a tau repeat domain can be operably linked to a polynucleotide encoding a reporter via a linker. In an embodiment, polynucleotides described herein can further comprise a polynucleotide encoding a linker.

Linkers

Methods for attaching two individual elements can require the use of a linker to create a bond between two molecules thought to be conjugated or fused to one another. Fusion proteins result from the fusion two or more protein domains together, and each protein or protein domain can be fused to the next using a linker. Suitable linkers for the fusion of two or more protein or protein domains can include natural linkers, and empirical linkers.

Natural linkers can be derived from multi-domain proteins, which are naturally present between protein domains. Natural linkers can have several properties depending or their such as length, hydrophobicity, amino acid residues, and secondary structure, which can impact the fusion protein in different way.

Many empirical linkers with various sequences and conformations can be used for the construction of recombinant fusion proteins. Empirical linkers can be classified in three types: flexible linkers, rigid linkers, and cleavable linkers. Flexible linkers can provide a certain degree of movement or interaction at the joined domains. They are generally composed of small, non-polar (e.g., Gly) or polar (e.g., Ser or Thr) amino acids, which provides flexibility, and allows for mobility of the connecting functional domains. Rigid linkers can successfully keep a fixed distance between the domains to maintain their independent functions, which can provide efficient separation of the protein domains or sufficient reduction of their interference with each other. Cleavable linkers can allow the release of functional domains in vivo. By taking advantage of unique in vivo processes, they can be cleaved under specific conditions such as the presence of reducing reagents or proteases. This type of linker can reduce steric hindrance, improve bioactivity, or achieve independent actions/metabolism of individual domains of recombinant fusion proteins after linker cleavage. Non-limiting examples of empiric linkers can include those listed in Table 1.

TABLE 1
Examples of empiric linkers

| | Linker | | |
|---|---|---|---|
| Linker Function | Type | Sequence | SEQ ID NO: |
| Increase | flexible | (GGGGS)₁ | SEQ ID NO: 8 |
| Stability/Folding | flexible | (GGGGS)₂ | SEQ ID NO: 9 |
| | flexible | (GGGGS)₃ | SEQ ID NO: 10 |
| | flexible | (Gly)₈ | SEQ ID NO: 11 |
| | flexible | (Gly)₆ | SEQ ID NO: 12 |
| | rigid | (EAAAK)₃ | SEQ ID NO: 13 |
| | rigid | (EAAAK)ₙ (n = 1-3) | SEQ ID NO: 14-15 |
| Increase | rigid | A(EAAAK)₄ALEA(EAAAK)₄A | SEQ ID NO: 16 |
| expression | | | |
| Improve | flexible | (GGGGS)₃ | SEQ ID NO: 10 |
| biological | rigid | A(EAAAK)₄ALEA(EAAAK)₄A | SEQ ID NO: 16 |
| activity | flexible | GGGGS | SEQ ID NO: 8 |
| | rigid | PAPAP | SEQ ID NO: 17 |
| | rigid | AEAAAKEAAAKA | SEQ ID NO: 18 |
| | flexible | (GGGGS)ₙ (n = 1, 2, 4) | SEQ ID NO: 19 |
| | rigid | (Ala-Pro)ₙ (10-34 aa) | SEQ ID NO: 20-32 |
| | | (n = 5-17) | |
| | cleavable | disulfide | N/A |
| | cleavable | disulfide | N/A |
| Enable targeting | cleavable | VSQTSKLTR↓AETVFPDV^{b} | SEQ ID NO: 33 |
| | cleavable | PLG↓LWA^{c} | SEQ ID NO: 34 |
| | cleavable | RVL↓AEA; EDVVCC↓SMSY; | SEQ ID NO: 35 |
| | | GGIEGR↓GS^{c} | SEQ ID NO: 36 |
| | cleavable | TRHRQPR↓GWE; | SEQ ID NO: 37 |
| | | AGNRVRR↓SVG; | SEQ ID NO: 38 |
| | | RRRRRRR↓R↓R^{d} | SEQ ID NO: 39 |
| | cleavable | GFLG↓^{e} | SEQ ID NO: 40 |
| Alter PK | dipeptide | LE | |
| | rigid | A(EAAAK)₄ALEA(EAAAK)₄A | SEQ ID NO: 16 |
| | cleavable | Disulfide | N/A |

In an embodiment, a polynucleotide encoding a linker can comprise SEQ ID NO:5.

For example, polynucleotides can comprise a polynucleotide encoding a tau repeat domain comprising SEQ ID NO:1, a polynucleotide encoding a linker comprising SEQ ID NO:5, and a polynucleotide encoding a reporter comprising SEQ ID NO:2; or a polynucleotide can comprise a polynucleotide encoding a tau repeat domain comprising SEQ ID NO:1, a polynucleotide encoding a linker comprising SEQ ID NO:5, and a polynucleotide encoding a reporter comprising SEQ ID NO:3.

In another example, polynucleotides can encode a promoter comprising SEQ ID NO:4, a tau repeat domain comprising SEQ ID NO:1, and a linker comprising SEQ ID NO:5, and a reporter comprising SEQ ID NO:2; or a polynucleotide can encode a promoter comprising SEQ ID NO:4, a tau repeat domain comprising SEQ ID NO:1, a linker comprising SEQ ID NO:5, and a reporter comprising SEQ ID NO:3.

Polynucleotides encoding a promoter, a tau repeat domain, a linker, a reporter, or any combination thereof can be incorporated into an expression cassette. Polynucleotides encoding tau repeat domain can be operably linked to a promoter, for their own expression, or operably linked to a polynucleotide encoding a reporter via a linker, for the expression of a fusion protein comprising the tau repeat domain and the reporter. In an embodiment, a tau repeat domain (or seed tau protein) can be operably linked to a fluorescent protein that is a member of a proximity detection pair.

Vectors

An embodiment provides a vector comprising an expression cassette comprising a polynucleotide encoding a tau repeat domain and a reporter.

Expression Cassettes

A recombinant construct is a polynucleotide having heterologous polynucleotide elements. Recombinant constructs include expression cassettes or expression constructs, which refer to an assembly that is capable of directing the expression of a polynucleotide or gene of interest. An expression cassette generally includes regulatory elements such as a promoter that is operably linked to (so as to direct transcription of) a polynucleotide and often includes a polyadenylation sequence as well.

An expression cassette can comprise a fragment of DNA comprising a coding sequence of a selected polypeptide (e.g., a tau repeat domain) and regulatory elements preceding (5′ non-coding sequences) and following (3′ non-coding sequences) the coding sequence that are required for expression of the selected gene product. Thus, an expression cassette can comprise, for example: 1) a promoter sequence; 2) one or more coding sequences [“ORF”] (e.g., a tau repeat domain); and, 3) a 3′ untranslated region (i.e., a terminator) that, in eukaryotes, usually contains a polyadenylation site. Expression cassettes can be circular or linear nucleic acid molecules. Polynucleotides, expression cassettes, vectors, etc. as described herein can comprise one or more tau repeat domains. For example, a polynucleotide, expression cassette, or vector can comprise 1, 2, 3, 4, 5, 6 or more a tau repeat domains. The one or more tau repeat domains can be operably linked to one another, or separated out throughout the polypeptide, expression cassette, or vector.

A recombinant construct or expression cassette can be contained within a vector, to facilitate cloning and transformation. In addition to the components of the recombinant construct, the vector can include, one or more selectable markers, a signal which allows the vector to exist as single-stranded DNA (e.g., a M13 origin of replication), at least one multiple cloning site, and a origin of replication (e.g., a SV40 or adenovirus origin of replication). Different expression cassettes can be transformed into different organisms including bacteria, yeast, plants, and mammalian cells, as long as the correct regulatory elements are used for each host.

Generally, a polynucleotide or gene that is introduced into a genetically engineered organism is part of a recombinant construct. A polynucleotide can comprise a gene of interest, e.g., a coding sequence for a tau repeat domain, or can be a sequence that is capable of regulating expression of a gene, such as a regulatory element, an antisense sequence, a sense suppression sequence, or a miRNA sequence. A recombinant construct can include, for example, regulatory elements operably linked 5′ or 3′ to a polynucleotide encoding one or more polypeptides of interest. For example, a promoter can be operably linked with a polynucleotide encoding one or more polypeptides of interest (e.g., a tau repeat domain) when it is capable of affecting the expression of the polynucleotide (i.e., the polynucleotide is under the transcriptional control of the promoter). Polynucleotides can be operably linked to regulatory elements in sense or antisense orientation. The expression cassettes or recombinant constructs can additionally contain a 5′ leader polynucleotide. A leader polynucleotide can contain a promoter as well as an upstream region of a gene. The regulatory elements (i.e., promoters, enhancers, transcriptional regulatory regions, translational regulatory regions, and translational termination regions) and/or the polynucleotide encoding a signal anchor can be native/analogous to the host cell or to each other. Alternatively, the regulatory elements can be heterologous to the host cell or to each other. See, U.S. Pat. No. 7,205,453 and U.S. Patent Application Publication Nos. 2006/0218670 and 2006/0248616. An expression cassette or recombinant construct can additionally contain one or more selectable marker genes.

Methods for preparing polynucleotides operably linked to a regulatory element and expressing polypeptides in a host cell are well-known in the art. See, e.g., U.S. Pat. No. 4,366,246. A polynucleotide can be operably linked when it is positioned adjacent to or close to one or more regulatory elements, which direct transcription and/or translation of the polynucleotide.

In an embodiment a vector can comprise an expression cassette comprising a polynucleotide encoding a tau repeat domain and a reporter.

In an embodiment, a polynucleotide can comprise a polynucleotide encoding a tau repeat domain comprising SEQ ID NO:1. In another embodiment, a polynucleotide encoding a reporter can comprise SEQ ID NO: 2 or SEQ ID NO:3.

An expression cassette can comprise a polynucleotide encoding a tau repeat domain comprising SEQ ID NO:1 and a polynucleotide encoding a reporter comprising SEQ ID NO:2; or an expression cassette can comprise a polynucleotide encoding a tau repeat domain comprising SEQ ID NO:1 and a polynucleotide encoding a reporter comprising SEQ ID NO:3.

In an embodiment, an expression cassette can further comprise a polynucleotide comprising a promoter and a promoter comprising SEQ ID NO:4.

For example, an expression cassette can comprise a polynucleotide encoding a promoter comprising SEQ ID NO:4, a polynucleotide encoding a tau repeat domain comprising SEQ ID NO:1, and a polynucleotide encoding a reporter comprising SEQ ID NO:2; or an expression cassette can comprise a polynucleotide encoding a promoter comprising SEQ ID NO:4, a polynucleotide encoding a tau repeat domain comprising SEQ ID NO:1, and a polynucleotide encoding a reporter comprising SEQ ID NO:3.

In an embodiment, an expression cassette can further comprise a polynucleotide comprising a linker and a polynucleotide encoding a linker, which can comprise SEQ ID NO:5.

For example, an expression cassette can comprise a polynucleotide encoding a polynucleotide encoding a promoter comprising SEQ ID NO:4, a tau repeat domain comprising SEQ ID NO:1, a polynucleotide encoding a linker comprising SEQ ID NO:5, and a polynucleotide encoding a reporter comprising SEQ ID NO:2; or an expression cassette can comprise a polynucleotide encoding a promoter comprising SEQ ID NO:4, a polynucleotide encoding a tau repeat domain comprising SEQ ID NO:1, a polynucleotide encoding a linker comprising SEQ ID NO:5, and a polynucleotide encoding a reporter comprising SEQ ID NO:3.

Vectors

An expression cassette can be delivered to cells (e.g., a plurality of different cells or cell types including target cells or cell types and/or non-target cell types) in a vector (e.g., an expression vector). A vector can be an integrating or non-integrating vector, referring to the ability of the vector to integrate the expression cassette and/or polynucleotide into a genome of a cell. Either an integrating vector or a non-integrating vector can be used to deliver an expression cassette containing one or more polypeptides described herein. Examples of vectors include, but are not limited to, (a) non-viral vectors such as nucleic acid vectors including linear oligonucleotides and circular plasmids; artificial chromosomes such as human artificial chromosomes (HACs), yeast artificial chromosomes (YACs), and bacterial artificial chromosomes (BACs or PACs); episomal vectors; transposons (e.g., PiggyBac); and (b) viral vectors such as retroviral vectors, lentiviral vectors, adenoviral vectors, and AAV vectors. Viruses have several advantages for delivery of nucleic acids, including high infectivity and/or tropism for certain target cells or tissues. In some cases, a virus is used to deliver a nucleic acid molecule or expression cassette comprising one or more regulatory elements, as described herein, operably linked to a gene.

In an embodiment, the vector is a lentiviral vector. Lentiviral vectors rely on Lentivirus for the infection and incorporation of genetic material into a host cell. Lentivirus is a genus of retroviruses characterized by long incubation periods. The best-known lentivirus is the human immunodeficiency virus (HIV), which causes AIDS. Lentiviruses can integrate a significant amount of DNA into the DNA of the host cell and can efficiently infect nondividing cells, so they are one of the most efficient methods of gene delivery. Lentiviruses can become endogenous, integrating their genome into the host germline genome, so that the virus is henceforth inherited by the host's daughter cells during cellular division. Lentiviral infection has advantages over other viral and non-viral vectors, including high-efficiency infection of dividing and nondividing cells, long-term stable expression of a transgene, and low immunogenicity. Non-limiting examples of lentiviral vectors include vector derived from lentiviruses such as human immunodeficiency virus (HIV), simian immunodeficiency virus (SIV) and feline immunodeficiency virus (FIV),

Vectors for stable transformation of mammalian are well known in the art and can be obtained from commercial vendors or constructed from publicly available sequence information. Expression vectors can be engineered to produce protein(s) of interest (e.g., tau repeat domain). Such vectors are useful for recombinantly producing a protein of interest and for modifying the natural phenotype of host cells.

If desired, polynucleotides can be cloned into an expression vector comprising expression control elements, including for example, origins of replication, promoters, enhancers, or other regulatory elements that drive expression of the polynucleotides in host cells. An expression vector can be, for example, a plasmid, such as pBR322, pUC, or ColE1, or an adenovirus vector, such as an adenovirus Type 2 vector or Type 5 vector. Optionally, other vectors can be used, including but not limited to Sindbis virus, simian virus 40, alphavirus vectors, poxvirus vectors, and cytomegalovirus and retroviral vectors, such as murine sarcoma virus, mouse mammary tumor virus, Moloney murine leukemia virus, and Rous sarcoma virus. Mini-chromosomes such as MC and MC1, bacteriophages, phagemids, yeast artificial chromosomes, bacterial artificial chromosomes, virus particles, virus-like particles, cosmids (plasmids into which phage lambda cos sites have been inserted) and replicons (genetic elements that are capable of replication under their own control in a cell) can also be used.

To confirm the presence of recombinant polynucleotides or recombinant genes in transgenic cells, a polymerase chain reaction (PCR) amplification or Southern blot analysis can be performed using methods known to those skilled in the art. Expression products of the recombinant polynucleotides or recombinant genes can be detected in any of a variety of ways, and include for example, western blot and enzyme assay. Once recombinant organisms have been obtained, they may be grown in cell culture.

Techniques contemplated herein for gene expression in mammalian cells can include delivery via a viral vector (e.g., retroviral, adenoviral, AAV, helper-dependent adenoviral systems, hybrid adenoviral systems, herpes simplex, pox virus, lentivirus, and Epstein-Barr virus), and non-viral systems, such as physical systems (naked DNA, DNA bombardment, electroporation, hydrodynamic, ultrasound, and magnetofection), and chemical system (cationic lipids, different cationic polymers, and lipid polymers).

For example, vectors described herein can be introduced into a cell to be altered thus allowing expression of the recombinant protein using any of the variety of methods that are known in the art and suitable for introduction of nucleic acid molecule into a cell. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion. Other methods of transfection include proprietary transfection reagents such as Lipofectamine™, Dojindo Hilymax™, Fugene™, jetPEI™ Effectene™ and DreamFect™.

The procedures described herein employ, unless otherwise indicated, conventional techniques of chemistry, molecular biology, microbiology, recombinant DNA, genetics, immunology, cell biology, cell culture and transgenic biology, which are within the skill of the art. (See, e.g., Maniatis, et al., Molecular Cloning, Cold Spring Harbor Laboratory Press, Cold Spring Harbor, N.Y. (1982); Sambrook et al., (1989); Sambrook and Russell, Molecular Cloning, 3rd Ed., Cold Spring Harbor Laboratory Press, Cold Spring Harbor, N.Y. (2001); Ausubel, et al., Current Protocols in Molecular Biology, John Wiley & Sons (including periodic updates) (1992); Glover, DNA Cloning, IRL Press, Oxford (1985); Russell, Molecular biology of plants: a laboratory course manual, Cold Spring Harbor Laboratory Press, Cold Spring Harbor, N.Y. (1984); Anand, Techniques for the Analysis of Complex Genomes, Academic Press, NY (1992); Guthrie and Fink, Guide to Yeast Genetics and Molecular Biology, Academic Press, NY (1991); Harlow and Lane, Antibodies, Cold Spring Harbor Laboratory Press, Cold Spring Harbor, N.Y. (1988); Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, A. R. Liss, Inc. (1987); Immobilized Cells And Enzymes, IRL Press (1986); B. Perbal, A Practical Guide To Molecular Cloning (1984); the treatise, Methods In Enzymology, Academic Press, Inc., NY); Methods In Enzymology, Vols. 154 and 155, Wu, et al., eds.; Immunochemical Methods In Cell And Molecular Biology, Mayer and Walker, eds., Academic Press, London (1987); Handbook Of Experimental Immunology, Volumes I-IV, D. M. Weir and C. C. Blackwell, eds. (1986); Riott, Essential Immunology, 6th Edition, Blackwell Scientific Publications, Oxford (1988); Fire, et al., RNA Interference Technology From Basic Science to Drug Development, Cambridge University Press, Cambridge (2005); Schepers, RNA Interference in Practice, Wiley-VCH (2005); Engelke, RNA Interference (RNAi): The Nuts & Bolts of siRNA Technology, DNA Press (2003); Gott, RNA Interference, Editing, and Modification: Methods and Protocols (Methods in Molecular Biology), Human Press, Totowa, N.J. (2004); and Sohail, Gene Silencing by RNA Interference: Technology and Application, CRC (2004)).

In an embodiment, a vector can comprise an expression cassette comprising a polynucleotide encoding a tau repeat domain and a polynucleotide encoding a reporter. A polynucleotide encoding a tau repeat domain can, for example, comprise a sequence as set forth in SEQ ID NO:1. A polynucleotide encoding a reporter can, for example, comprise a sequence as set forth in SEQ ID NO:2 or SEQ ID NO:3.

In another embodiment, a vector can further comprise a polynucleotide encoding a promoter. A polynucleotide encoding a promoter can, for example, comprise a sequence as set forth in SEQ ID NO:4.

In an additional embodiment, a vector can further comprise a polynucleotide encoding a linker. A polynucleotide encoding a linker can, for example, comprise a sequence as set forth in SEQ ID NO:5.

Polynucleotides within a vector can be operably linked to one another. For example, the vector can comprise a polynucleotide encoding a promoter comprising SEQ ID NO:4, a tau repeat domain comprising SEQ ID NO:1, a polynucleotide encoding a linker comprising SEQ ID NO:5, and a polynucleotide encoding a reporter comprising SEQ ID NO:2 in operable linkage. In an embodiment, a vector can comprise SEQ ID NO:6.

In another example, a vector can comprise a polynucleotide comprising SEQ ID NO:4, a polynucleotide encoding a tau repeat domain comprising SEQ ID NO:1, a polynucleotide encoding a linker comprising SEQ ID NO:5, and a polynucleotide encoding a reporter comprising SEQ ID NO:3 in operable linkage. In an embodiment, a vector can comprise SEQ ID NO:7.

Vectors comprising polynucleotides molecules as described herein, encoding a tau repeat domain a report, a promoter, a linker, or any combination thereof can be incorporated into host cells for expression of the encoded polypeptides.

Cells

An embodiment provides a cell comprising: (i) a first vector comprising a polynucleotide encoding a tau repeat domain and a first reporter, and a second vector comprising a polynucleotide encoding a tau repeat domain and a second reporter; or (ii) a vector comprising a first polynucleotide encoding a tau repeat domain and a first reporter, and a second polynucleotide encoding a tau repeat domain and a second reporter.

Vectors described herein can be introduced into host cell (or more generally cell) to be altered thus allowing expression of recombinant, heterologous polypeptides within the cell. A variety of cells are known in the art and suitable for recombinant proteins expression. Examples of typical cells used for transfection include, but are not limited to, a bacterial cell, a eukaryotic cell, a yeast cell, an insect cell, a mammalian cell or a plant cell. Non-limiting examples of cells can include, E. coli, Bacillus, Streptomyces, Pichia pastoris, Salmonella typhimurium, Drosophila S2, Spodoptera SJ9. A mammalian cell can include, for example a cell derived from a rodent (such as a mouse, a rat, or a hamster), a primate (such as a monkey, or a human). Mammalian cells can be derived from a healthy tissue, or from a diseased tissue such as a tumor. Mammalian cells can be immortalized, to ensure non-limiting cell growth in culture. Non-limiting examples of mammalian cells can include, CHO, COS (e.g., COS-7), 3T3-F442A, HeLa, HUVEC, HUAEC, NIH 3T3, Jurkat, Human Embryonic Kidney (HEK) 293, HEK293H, or HEK293F.

In an embodiment, the cell can be a HeLa cell. HeLa is an immortal cell line widely used in scientific research. It is the oldest and most commonly used human cell line, that was derived from cervical cancer cells in 1951. The cell line was found to be remarkably durable and prolific, as compared to cells cultured from other human cells, which would only survive for a few days.

In an embodiment, the cell can be a HEK 293 cell. HEK 293 cells are a specific cell line originally derived from human embryonic kidney cells grown in tissue culture. HEK 293 cells have been widely used in cell biology research for many years, because of their reliable growth and propensity for transfection. They are also used by the biotechnology industry to produce therapeutic proteins and viruses for gene therapy.

A cell can comprise one or more expression cassettes. A cell can comprise one or more vectors comprising one or more heterologous polynucleotides not present in a corresponding wild-type cell. In an embodiment, a cell does not naturally comprise the vectors or expression cassettes.

An embodiment provides a cell comprising a first vector comprising a polynucleotide encoding a tau repeat domain and a first reporter, and a second vector comprising a polynucleotide encoding a tau repeat domain and a second reporter. In both vectors, the tau repeat domains can be identical tau repeat domains. In other embodiments, a first vector can comprise a polynucleotide encoding a first tau repeat domain, and a second vector can comprise a polynucleotide encoding a second tau repeat domain, wherein the first and the second tau repeat domains are non-identical. For example, non-identical tau repeat domains can have different lengths, but can still co-assemble with one another (e.g., both can comprise a glycine-rich region involved in protein-protein interactions of tau protein).

In an embodiment, a first reporter can comprise SEQ ID NO:2 or SEQ ID NO:3. For example, a cell can comprise a first vector comprising a polynucleotide encoding a tau repeat domain (e.g., SEQ ID NO:1), and a polynucleotide encoding a first reporter (e.g., mClover3, or SEQ ID NO:2), and a second vector comprising a polynucleotide encoding a tau repeat domain (e.g., SEQ ID NO:1), and a polynucleotide encoding a second reporter (e.g., a mCerulean3, or SEQ ID NO:3). In one embodiment, a polynucleotide encoding a tau repeat domain (e.g., SEQ ID NO:1), can be operably linked to a polynucleotide encoding a first reporter (e.g., mClover3, or SEQ ID NO:2), and a polynucleotide encoding a tau repeat domain (e.g., SEQ ID NO:1), can be operably linked to a polynucleotide encoding a second reporter (e.g., a mCerulean3, or SEQ ID NO:3).

In an embodiment, a polynucleotide encoding a tau repeat domain and a polynucleotide encoding a reporter can be linked through a linker; and a linker can comprise SEQ ID NO:5. For example, a cell can comprise a first vector comprising a polynucleotide encoding a tau repeat domain (e.g., SEQ ID NO:1) linked to a polynucleotide encoding a first reporter (e.g., mClover3, or SEQ ID NO:2) via a linker (e.g., SEQ ID NO:5); and a second vector comprising a polynucleotide encoding a tau repeat domain (e.g., SEQ ID NO:1), linked to a polynucleotide encoding a second reporter (e.g., a mCerulean3, or SEQ ID NO:3) via a linker (e.g., SEQ ID NO:5).

In an embodiment, a polynucleotide encoding a tau repeat domain (operably linked to polynucleotide encoding a reporter, or linked to a polynucleotide encoding a reporter through a linker) can be operably linker to a promoter; and a promoter can comprise SEQ ID NO:4. For example, a cell can comprise a first vector comprising a polynucleotide encoding a promoter (e.g., SEQ ID NO:4), a polynucleotide encoding a tau repeat domain (e.g., SEQ ID NO:1), and a polynucleotide encoding a first reporter (e.g., mClover3, or SEQ ID NO:2); and a second vector comprising a polynucleotide encoding a promoter (e.g., SEQ ID NO:4), a polynucleotide encoding a tau repeat domain (e.g., SEQ ID NO:1), and a polynucleotide encoding a second reporter (e.g., a mCerulean3, or SEQ ID NO:3). The polynucleotides can be operably linked with one another.

In another example, a cell can comprise a first vector comprising a polynucleotide encoding a promoter (e.g., SEQ ID NO:4) operably linked to a polynucleotide encoding a tau repeat domain (e.g., SEQ ID NO:1) linked to a polynucleotide encoding a first reporter (e.g., mClover3, or SEQ ID NO:2) via a linker (e.g., SEQ ID NO:5); and a second vector comprising a polynucleotide encoding a promoter (e.g., SEQ ID NO:4) operably linked to a polynucleotide encoding a tau repeat domain (e.g., SEQ ID NO:1), linked to a polynucleotide encoding a second reporter (e.g., a mCerulean3, or SEQ ID NO:3) via a linker (e.g., SEQ ID NO:5).

A cell can comprise, for example, a first vector comprising a polynucleotide comprising SEQ ID NO:6 and a second vector comprising a polynucleotide as set forth in SEQ ID NO:7.

In another embodiment, a vector can comprise a first polynucleotide comprising a polynucleotide encoding a tau repeat domain and a polynucleotide encoding a first reporter, and a second polynucleotide comprising a polynucleotide encoding a tau repeat domain and a polynucleotide encoding a second reporter. First and second polynucleotides can be operably linked to one another.

In an embodiment, a first reporter can comprise SEQ ID NO:2 or SEQ ID NO:3. For example, a cell can comprise a vector comprising a first polynucleotide encoding a tau repeat domain (e.g., SEQ ID NO:1), and a polynucleotide encoding a first reporter (e.g., mClover3, or SEQ ID NO:2), and a second polynucleotide encoding a tau repeat domain (e.g., SEQ ID NO:1), and a polynucleotide encoding a second reporter (e.g., a mCerulean3, or SEQ ID NO:3). In one embodiment, a polynucleotide encoding a tau repeat domain (e.g., SEQ ID NO:1), can be operably linked to a first reporter (e.g., mClover3, or SEQ ID NO:2), and a polynucleotide encoding a tau repeat domain (e.g., SEQ ID NO:1), can be operably linked to second reporter (e.g., a mCerulean3, or SEQ ID NO:3).

In an embodiment, a polynucleotide encoding a tau repeat domain and a polynucleotide encoding a reporter can be linked through a linker; and a linker can comprise SEQ ID NO:5. For example, a cell can comprise a vector comprising a first polynucleotide encoding a tau repeat domain (e.g., SEQ ID NO:1) linked to a polynucleotide encoding a first reporter (e.g., mClover3, or SEQ ID NO:2) via a linker (e.g., SEQ ID NO:5); and a second polynucleotide encoding a tau repeat domain (e.g., SEQ ID NO:1), linked to a polynucleotide encoding a second reporter (e.g., a mCerulean3, or SEQ ID NO:3) via a linker (e.g., SEQ ID NO:5).

In an embodiment, a polynucleotide encoding a tau repeat domain (operably linked to polynucleotide encoding a reporter, or linked to a polynucleotide encoding a reporter through a linker) can be operably linker to a promoter; and a promoter can comprise SEQ ID NO:4. For example, a cell can comprise a vector comprising a first polynucleotide encoding a promoter (e.g., SEQ ID NO:4), a polynucleotide encoding a tau repeat domain (e.g., SEQ ID NO:1), and a polynucleotide encoding a first reporter (e.g., mClover3, or SEQ ID NO:2); and a second polynucleotide encoding a promoter (e.g., SEQ ID NO:4), a polynucleotide encoding a tau repeat domain (e.g., SEQ ID NO:1), and a polynucleotide encoding a second reporter (e.g., a mCerulean3, or SEQ ID NO:3). The polynucleotides can be operably linked with one another.

In another example, a cell can comprise a vector comprising a first polynucleotide encoding a promoter (e.g., SEQ ID NO:4) operably linked to a polynucleotide encoding a tau repeat domain (e.g., SEQ ID NO:1) linked to a first reporter (e.g., mClover3, or SEQ ID NO:2) via a linker (e.g., SEQ ID NO:5); and a second polynucleotide comprising a polynucleotide encoding a promoter (e.g., SEQ ID NO:4) operably linked to a polynucleotide encoding a tau repeat domain (e.g., SEQ ID NO:1), linked to second reporter (e.g., a mCerulean3, or SEQ ID NO:3) via a linker (e.g., SEQ ID NO:5).

In an additional embodiment, a polynucleotide encoding a tau repeat domain (operably linked to polynucleotide encoding a reporter, or linked to a polynucleotide encoding a reporter through a linker) can be operably linked to a promoter; and a promoter can comprise SEQ ID NO:4. For example, a cell can comprise a vector comprising a first polynucleotide encoding a promoter (e.g., SEQ ID NO:4), a polynucleotide encoding a tau repeat domain (e.g., SEQ ID NO:1), and a first reporter (e.g., mClover3, or SEQ ID NO:2); and a second polynucleotide encoding a polynucleotide encoding a tau repeat domain (e.g., SEQ ID NO:1), and a polynucleotide encoding a second reporter (e.g., a mCerulean3, or SEQ ID NO:3) via a linker (e.g., SEQ ID NO:5). The polynucleotides sequences can be operably linked with one another.

In another example, a cell can comprise a vector comprising a first polynucleotide encoding a promoter (e.g., SEQ ID NO:4) operably linked to a polynucleotide encoding a tau repeat domain (e.g., SEQ ID NO:1) linked to a first reporter (e.g., mClover3, or SEQ ID NO:2) via a linker (e.g., SEQ ID NO:5); and a second polynucleotide encoding a polynucleotide encoding a tau repeat domain (e.g., SEQ ID NO:1), linked to a polynucleotide encoding a second reporter (e.g., a mCerulean3, or SEQ ID NO:3) via a linker (e.g., SEQ ID NO:5).

Polynucleotides and expression cassettes described herein can be incorporated into lentiviral expression vectors for the transduction of cells. Single cell colonies can then be selected for characterization, and resulting clones having variable tau expression can be picked. For example, cells as described herein, transduced with vectors described herein can be selected based on several characteristics, such as the minimal background FRET, the strongest induced response to exogenous seed tau protein, and overall tau protein expression. a version with low-expressing clone (v2L) levels of tau can, for example, be obtained. Such cells have been deposited at ATCC as Tau RD(P301S)v2L biosensors.

In an embodiment, the cell can express Tau RD(P301S).

Methods of Identifying Seed Tau Protein in a Sample

An embodiment provides a method of measuring a titer of or of detecting seed tau protein in a sample.

Tau protein forms self-replicating assemblies (seeds) that may underlie progression of pathology in Alzheimer's disease (AD) and related tauopathies. The present disclosure relies on the demonstration that seeding in recombinant protein preparations and brain homogenates can be quantified with biosensor cell lines that express tau with a disease-associated mutation (e.g., P301S) fused to complementary reporter proteins. Quantification of induced aggregation in cells that score positive by fluorescence resonance energy transfer (FRET) can accomplished by cell imaging or flow cytometry, for example. biosensor cells described herein can be about 50, 100, 200, 300, 400, 500-fold or more sensitive than available biosensor cell lines, and when coupled with immunoprecipitation can reliably detect seeding at attomolar levels (e.g., about 1, 10, 20, 30, 40, 50, 60, 70, 80, 90, 100, 200, 300, 400, 500, 600 attomolar or more) of recombinant tau fibrils or about 1, 5, 10, 30, 30, 40, 50, 60, 70, 80, 90, 100, 200, 300, 400, 500 pg/ml or more of AD brain homogenate.

In an embodiment, a sample can be contacted with a cell comprising one or more polynucleotides, expression cassettes, or vectors described herein. A seeding assay can be performed; and a titer or a detection of seed tau protein can be detected, for example by flow cytometry, in the sample.

As used herein, a method of measuring a titer of or of detecting a seed tau protein relies on the realization of a seeding assay. The term “seeding assay” generally refers to the realization of fluorescence resonance energy transfer (FRET) on cells as described herein, cultured under conditions to express seed tau proteins that can aggregate, such that the aggregation of seed tau protein fused or linked to a different reporter can be brought into close contact, and such close contact can be detected by FRET. In the context of a seeding assay, the different reporters (e.g., the first and the second reporter) can also be referred to as a donor fluorescent protein and an acceptor fluorescent protein.

A cell can be cultured under any suitable culture conditions and contacted with a test sample. A cell can be exposed to a laser or other suitable light source producing an excitation light corresponding to the excitation wavelength of a donor fluorescent protein (e.g., a first reporter). For example, a laser or other suitable light source producing an excitation light between 485-588 nm can be used. Light can then be collected at a wavelength corresponding to the emission wavelength of the acceptor fluorescent protein (e.g., a second reporter), which corresponds to the emission light signal. For example, light can be collected between 500-670 nm for the detection of emission light signal.

Depending on the technology used to collect emission light signals (fluorescent microscopy, flow cytometry, or other suitable method for example), several images can be taken of the cells, and/or multiple cells can be analyzed.

A donor fluorescent protein, in an excited state energy can transfer energy directly to an acceptor fluorescent protein in close proximity without emitting a photon. The resulting fluorescence sensitized emission has characteristics similar to the emission spectrum of the acceptor and can be detected by immunofluorescent microscopy or by flow cytometry, for example. Any other suitable means for the detection of fluorescence can also be used.

A cell comprising one or more polynucleotides, expression cassettes, vectors described herein can express a seed tau protein linked or fused to a donor fluorescent protein and a seed tau protein linked or fused to an acceptor fluorescent protein. The seed tau proteins can aggregate with one another to form tau protein aggregates, which can bring a donor fluorescent protein in close proximity to an acceptor fluorescent protein. Upon exposition of such cell to an excitation light, energy from a donor fluorescent protein can be transferred to an acceptor fluorescent protein, which can emit emission light signal that can be detected.

In the presence of exogenous tau protein, tau protein fragment or aggregates thereof in a sample (i.e., tau proteins, fragments or aggregates not expressed or generated by a reporter cell), for example providing from a sample comprising tau proteins, fragments or aggregates, seed tau protein linked to a fluorescent protein, expressed by a reporter cell, can interact with and form aggregates with exogenous tau proteins, fragments or aggregates. Exogenous tau proteins, fragments or aggregates can compete with seed tau protein linked to fluorescent proteins (either donor or acceptor), and generate aggregates comprising exogenous tau proteins, fragments or aggregates and seed tau protein linked to a fluorescent protein. A binding competition can result in the generation of a distance between a donor fluorescent protein and an acceptor fluorescent protein, which can prevent an energy transfer from a donor fluorescent protein to an acceptor fluorescent protein, resulting in a reduction or in a lack of emission of a light signal. Therefore, detecting an emission light signal can indicate that a sample does not comprise tau protein, fragment or aggregate, while a lack of or a decrease of an emission light signal can indicate that a sample comprises tau proteins, fragments or aggregates.

An emission light signal measured in the absence of a sample, or in the presence of a sample known for not containing any tau protein can be used as a negative control. In such case, nothing disturbs the interaction between a seed tau protein linked to a donor fluorescent protein and a seed tau protein linked to an acceptor fluorescent protein; and an emission light signal can be detected.

An emission light signal measured in a reporter cell that does not express a seed tau protein linked to a donor fluorescent protein nor a seed tau protein linked to an acceptor fluorescent protein (i.e., a cell expressing a seed tau protein linked to a donor fluorescent protein only, a cell expressing a seed tau protein linked to an acceptor fluorescent protein only, or a cell not expressing any seed tau protein) can be used as an internal control, to evaluate any auto-fluorescent signal that can be emitted by a cell. In such case, no emission light signal can be detected as a result of a transfer of energy from a donor fluorescent protein to an acceptor fluorescent protein; and nothing but cell autofluorescence can be detected.

Furthermore, an emission light signal measured in the presence of a sample comprising exogenous tau proteins, fragments or aggregates can be used as a positive control. In such case, exogenous tau protein, fragment or aggregate can disturb the interaction between a seed tau protein linked to a donor fluorescent protein and a seed tau protein linked to an acceptor fluorescent protein; and a weaker or absent emission light signal can be detected, as compared to a negative control, and the decrease in the emission signal can be proportionate to the amount of exogenous tau protein, fragment or aggregate present.

If an emission light signal measured in a sample is equivalent to or greater than a negative control, it can indicate that a sample does not comprise tau protein, fragment or aggregate.

If an emission light signal measured in a sample is less than a negative control, or greater than a positive control, it can indicate that a sample does comprise tau proteins, fragments or aggregates. Alternatively, if an emission light measured in a sample is greater than or equivalent to a positive control, it can indicate that a sample does comprise tau proteins, fragments or aggregates. Similarly, if an emission light measured in a sample is less than or equivalent to a negative control and greater than an internal control, it can indicate that a sample does comprise tau proteins, fragments or aggregates.

If an emission light signal measured in a sample is less than an internal control, it can indicate that a test is inconclusive, and no conclusion can be reached regarding the presence or absence of tau proteins, fragments or aggregates in a sample.

Binding competition can be weak, if an amount of exogenous tau protein, fragment or aggregate from a sample is small; and can be strong, if an amount of exogenous tau protein, fragment, or aggregate from a sample is large. An amount of exogenous tau protein, fragment, or aggregates can thus modify an amount of emission light signal detected in a dose dependent manner, which can be used to measure a titer to tau protein, fragment, or aggregate in a sample. The emission light signal can be compared to a standard curve comparing emission light signals obtained in the presence of predetermined amounts of tau protein. For example, emission light signals measured in the presence of various concentrations of tau protein, fragment, or aggregates can be used to generate a standard curve.

If an emission light signal measured in a sample is equivalent to or greater than a negative control, it can indicate that the titer of tau proteins, fragments or aggregates in the sample is zero.

If an emission light signal measured in a sample is less than a negative control, but greater than a positive control, it can then be compared to the emission light signal in a standard curve, to estimate the concentration (i.e., the titer) of tau proteins, fragments or aggregates present in the sample.

The methods described herein can be used to detected tau proteins at the attomolar level. To achieve such sensitivity the method can include an immunoprecipitation step, to further concentrate the tau protein collected from a sample, prior to contacting the sample with the cells.

In an embodiment, tau protein present in the sample can be immunoprecipitated prior to contacting the sample with the cells described herein. For example, beads can be incubated with an antibody against a microtubule-binding repeat of tau, a biological sample can be incubated with the bead, and an enriched tau suspension of the sample can be obtained by elution of the beads. In an embodiment, about 10-200 μl of beads can be incubated with an anti-tau repeat domain. For example, 10, 20, 30, 40, 50, 60, 70, 80, 90, 100, 110, 120, 130, 140, 150, 160, 170, 180, 190 or 200 μl of beads can be used. In another embodiment, about 1-20 ml of a biological sample can be incubated with the beads. For example, 1, 2, 3, 4, 5, 6, 7, 8, 9, 10, 15, or 20 ml of CSF can be incubated with the beads. The antibody can be any antibody capable of specifically recognizing tau binding domains. The antibody can be a monoclonal antibody or a polyclonal antibody.

As used herein, a “sample” refers to any biological material such as a biological fluid, a tissue sample, a tissue homogenate, and the like that can be collected from a subject, and that is susceptible to contains tau proteins, fragments or aggregates, and that could be used in the methods described herein.

In an embodiment, the sample can be a biological fluid, a tissue sample, a cerebrospinal fluid, a brain homogenate, or an aggregated material amplified in vitro therefrom.

An additional embodiment provides a method of detecting attomolar levels of a seed tau protein in a sample comprising: (a) contacting the sample with the cells described herein; (b) performing a seeding assay; and (c) detecting tau protein aggregates by flow cytometry.

Methods of Detecting Neurodegenerative Tauopathy Disease or Condition in a Subject

Another embodiment provides a method of detecting Alzheimer's disease (AD), or a neurodegenerative tauopathy disease or condition linked to tau protein aggregation in a subject comprising: contacting a sample with the sensor cells described herein; performing a seeding assay; and detecting tau protein aggregates by, for example, flow cytometry, thereby detecting AD or neurodegenerative tauopathy disease or condition in a subject.

The method described herein can comprise culturing sensor cells described herein, and plating sensor cells at a cell density in a cell culture plate or dish. A culture plate can be a well plate. For example, a well plate can be a 96-well plate, a 48-well plate, a 24-well plate, a 12-well plate, or a 6-well plate. The cell culture dish can be a cell culture dish of any size.

Prior to contacting a sample with a sensor cell, sensor cells can be plated in a cell culture well or dish. A cell density for plating cells can be adjusted on the size of the well or dish. For example, a cell density can be about 5,000, 10,000, 20,000, 50,000, 100,000, 200,000 cells or more per well or per dish.

Sensor cells can be cultured at the cell density for a period of time prior to being contacted with a sample. For example, a period of time can be about 6, 12, 24, 26, 48, 72, or more hours.

A sensor cell can then be contacted with a sample, in the presence of in the absence of any agent that would facilitate or enhance the aggregation of tau protein present in the sample with seed tau protein expressed by the sensor cell. Such agent can be a cationic lipid reagent. For example, a cationic lipid reagent can be Lipofectamine 2000 transfection reagent.

Sensor cells can be separated from a cell culture dish or cell culture well and fixed prior to measuring a level of light signal emitted by a sensor cell. Sensor cells can be collected, after an exposure period. For example, an exposure period can be about 6, 12, 24, 36, 48, 72 or more hours after incubation of a sample with a sensor cell.

Sensor cells can be fixed to preserve and stabilize cell morphology; to inactivate proteolytic enzymes that could otherwise degrade the sample; to strengthen samples so that they can withstand further processing and staining; and to ensure that protein interactions remain intact. Various fixative agents can be used to fix cells. For example, a fixative agent can be 4% (w/v) Paraformaldehyde, 4% (w/v) Paraformaldehyde-1% (v/v), glutaraldehyde, 10% Neutral-buffered formalin (NBF), Bouin's fixative, Zenker's solution, Helly solution, Carnoy's solution, ice-cold acetone (100%) or methanol (100%), and 1% (w/v) osmium tetroxide. The choice of fixative and fixation protocol may depend on the additional processing steps and final analyses that are planned.

An emission light signal can be correlated with the presence of tau protein in a sample, and therefore with the detection of AD or of a neurodegenerative disease in a subject from which a sample has been collected.

As used herein, diseases and conditions that can be referred to as “neurodegenerative tauopathy diseases or conditions” can be characterized by the pathological accumulation of tau aggregates, which are responsible for neurodegeneration. Non-limiting examples of neurodegenerative tauopathy disease or condition can include Alzheimer's disease (AD), primary age-related tauopathy (PART)/Neurofibrillary tangle-predominant senile dementia, chronic traumatic encephalopathy (CTE), progressive supranuclear palsy (PSP), corticobasal degeneration (CBD), frontotemporal dementia and parkinsonism linked to chromosome 17 (FTDP-17), lytico-bodig disease (Parkinson-dementia complex of Guam), ganglioglioma and gangliocytoma, meningioangiomatosis, postencephalitic parkinsonism, subacute sclerosing panencephalitis (SSPE), lead encephalopathy, tuberous sclerosis, pantothenate kinase-associated neurodegeneration, and lipofuscinosis.

An embodiment provides methods for detecting neurodegenerative tauopathy disease or condition in a subject.

Tau protein fragments can aggregate with one another, accumulate, and spread using prion mechanisms of action. Tau protein fragments or aggregates (seed tau protein or tau protein prions) are pathological and can be detected in subjects diagnosed with neurodegenerative diseases, associated with the accumulation of pathological protein in neurons, responsible for neurodegeneration. Therefore, detecting tau protein fragments or aggregates in a sample collected from a subject, as detailed above, can be used to detect or diagnose a neurological disease or condition associated with the accumulation of tau protein fragments or aggregates, such as AD.

A “sample” or “test sample” can be collected from a subject, in which the presence of, or the titer of tau proteins, fragments or aggregates is sought to be measured. A “test sample” is a sample for which the presence (or absence) of or the titer of tau protein is sought to be analyzed. The sample can be a biological fluid, a tissue sample, or an aggregated material amplified in vitro therefrom. A biological fluid can be, for example, whole blood, plasma, serum, cerebrospinal fluid (CSF), interstitial fluid, urine, lymph, saliva, tear, or any other biological fluid susceptible to contain tau protein.

A sample can be prepared in any suitable way to facilitate, enhance, or improve the detection or measurement of tau proteins, fragments or aggregates. For example, a sample can be concentrated, or diluted; material present in the sample can be amplified (using a protein-prion amplification technique for example); proteins can be extracted from a sample, a sample can be homogenized or sonicated, immunoprecipitated, or combinations thereof.

The term “subject” as used herein can refer to any individual or patient to which the methods described herein can be performed, and specifically from whom a sample can be collected. Generally, the subject is human, although as will be appreciated by those in the art, the subject may be an animal. Thus, other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

A sample collected from a subject can be contacted with cells comprising one or more polynucleotides, expression cassettes, or vectors described herein. The cell can be exposed to an excitation light. An emission light signal can be detected. A tau protein or aggregate can be detected in the sample, and a neurodegenerative tauopathy related disease or condition can be detected in a subject.

For example, emission light signals can be compared to positive or negative controls as described above and/or to a standard curve as described above. In the case of a negative control nothing disturbs the interaction between a seed tau protein linked or fused to a donor fluorescent protein and a seed tau protein linked or fused to an acceptor fluorescent protein and an emission light signal can be detected. In the case of an internal control, no tau protein related light can be emitted, an auto-fluorescent signal that can be emitted by a cell can be detected, but no emission light signal can be detected as a result of a transfer of energy from a donor fluorescent protein to an acceptor fluorescent protein. In the case of a negative control exogenous tau protein, fragment or aggregate disturbs the interaction between a seed tau protein linked to a donor fluorescent protein and a seed tau protein linked to an acceptor fluorescent protein; and a lesser or no emission light signal can be detected.

If an emission light signal measured in a sample collected from a subject is equivalent to or greater than a positive control, it can indicate that a sample does not comprise tau protein, fragment or aggregate; and that the subject does not have a neurodegenerative tauopathy disease, or condition.

If an emission light signal measured in a sample collected from a subject is less than a positive control, or greater than a negative control, it can indicate that a sample comprises tau proteins, fragments, or aggregates; and that the subject has or is susceptible to a neurodegenerative tauopathy disease or condition. Alternatively, if an emission light measured in a sample collected from a subject is greater than or equivalent to a negative control, it can indicate that a sample comprises tau proteins, fragments or aggregates; and that the subject has or is susceptible to a neurodegenerative tauopathy disease or condition. Similarly, if an emission light measured in a sample is less than or equivalent to a negative control and greater than an internal control, it can indicate that a sample does comprise tau proteins, fragments or aggregates; and that the subject has a neurodegenerative tauopathy disease or condition.

If an emission light measured in a sample is less than an internal control, it can indicate that a test is inconclusive, and no conclusion can be reach regarding the presence or absence of tau proteins, fragments or aggregates in a sample; and therefore, regarding the detection of a neurodegenerative tauopathy disease or condition in a subject.

An emission light signal can be compared to a standard curve comparing emission light signals obtained in the presence of predetermined amounts of tau protein. For example, emission light signals measured in the presence of various concentrations of tau proteins or fragments can be used to generate a standard curve. If an emission light signal measured in a sample is less than a positive control, but greater than a negative control, it can then be compared to the emission light signal in a standard curve, to estimate the concentration (i.e., the titer) of tau proteins, fragments, or aggregates present in the sample.

In an embodiment, the cell (i.e., sensor cell) can detect about 1, 5, 10, 30, 30, 40, 50, 60, 70, 80, 90, 100, 200, 300, 400, 500 pg/ml or more of tau protein in the sample.

In another embodiment, the method can further comprise administering a tau protein aggregation inhibitor to a subject.

The methods described herein can be used to measure the titer of or detecting a seed tau protein in a sample collected from a subject, which can in turn be used to detect or diagnose a neurodegenerative tauopathy disease or condition in the subject. If a neurodegenerative tauopathy disease or condition is detected in a subject, a tau protein aggregation inhibitor can be administered to the subject to treat, reduce or lessen the symptoms, or to slow down the evolution of the disease.

By “tau protein aggregation inhibitor”, it is meant any small molecule, compound, drug, or the like that is capable of limiting or reducing the aggregation of tau protein with one another to generate tau protein aggregates, responsible for neurodegenerative tauopathy diseases and conditions.

Methods of Identifying a Tau Protein Aggregation Inhibitor

An embodiment provides a method of identifying a tau protein aggregation inhibitor.

Neurodegenerative disease and conditions are generally fatal, with few existing options to slow-down, halt, inhibit or even reverse the accumulation of pathological proteins responsible for the neurodegeneration. Therefore, identifying tau protein aggregation inhibitors, by assessing if a putative tau protein aggregation inhibitor can impact the detection of tau protein, fragment or aggregate in a sample (as detailed above), can be used to identify such inhibitors.

Cells comprising one or more polynucleotides, expression cassettes, and/or vectors as described herein can be contacted with one or more putative tau protein aggregation inhibitors, selected from a library of compounds for example. The cells can be exposed to an excitation light. An emission light signal can be detected. Tau protein aggregation, or lack thereof can be detected in the sample; and tau protein aggregation inhibitor can be identified.

The method described herein can comprise culturing sensor cells described herein, and plating sensor cells at a cell density in a cell culture plate or dish.

Sensor cells can be contacted with a putative tau protein aggregation inhibitor, in the presence of in the absence of a cationic lipid reagent, such as Lipofectamine 2000 transfection reagent, for example.

Sensor cells can be separated from a cell culture dish or cell culture well and fixed prior to measuring a level of light signal emitted by a sensor cell, which can be correlated with the presence of tau protein aggregate in a sample, and therefore with the identification of a putative tau protein aggregation inhibitor.

Detecting tau protein aggregates can indicate that the putative tau protein aggregation inhibitor does not inhibit tau protein aggregation. A lack of detection of tau protein aggregates can indicate that the putative tau protein aggregation inhibitor inhibits tau protein aggregation.

For example, an emission light signal measured in the absence of a test compound, or in the presence of a compound known for not being a tau protein aggregation inhibitor can be used as a positive control. In such case, nothing disturbs the interaction between a seed tau protein linked to a donor fluorescent protein and a seed tau protein linked to an acceptor fluorescent protein; and an emission light signal can be detected. An emission light signal measured in the presence of tau proteins, fragments, or aggregates, or in the presence of a compounds known for being a tau protein aggregation inhibitor can be used as a negative control. In such case, tau proteins, fragments, or aggregates, or a tau protein aggregation inhibitor can interact with seed tau protein linked to either a donor fluorescent protein or an acceptor fluorescent protein, thereby disturbing the interaction between a seed tau protein linked to a donor fluorescent protein and a seed tau protein linked to an acceptor fluorescent protein and generating a distance between them. An emission light signal measured in cells that do not express a seed tau protein linked to a donor fluorescent protein nor a seed tau protein linked to an acceptor fluorescent protein (i.e., a cell expressing a seed tau protein linked to a donor fluorescent protein only, a cell expressing a seed tau protein linked to an acceptor fluorescent protein only, or a cell not expressing any seed tau protein) can be used as an internal control, to evaluate any auto-fluorescent signal that can be emitted by a cell. In such case, no emission light signal can be detected as a result of a transfer of energy from a donor fluorescent protein to an acceptor fluorescent protein; and nothing but cell autofluorescence can be detected.

If an emission light signal measured in a sample comprising a test compound is equivalent to or greater than a positive control, it can indicate that a seed tau protein linked to a donor fluorescent protein can interact with a seed tau protein linked to an acceptor fluorescent protein in the sample, and that a test compound is not a tau protein aggregation inhibitor.

If an emission light signal measured in a sample comprising a test compound is equivalent or less than a negative control, or if an emission light signal is greater than a negative control and less than a positive control, it can indicate that a seed tau protein linked to a donor fluorescent protein cannot fully interact with a seed tau protein linked to an acceptor fluorescent protein in the sample, and that a test compound is a tau protein aggregation inhibitor.

If an emission light measured in a sample is less than an internal control, it can indicate that a test is inconclusive, and no conclusion can be reach regarding the presence or absence of tau proteins, fragments or aggregates in a sample, and therefore regarding the status of a compound as a tau protein aggregation inhibitor.

Methods of Identifying Tau Protein Aggregation Regulator or Modulator

An embodiment provides a method of identifying tau protein aggregation regulator or modulator.

As used herein, the term “tau protein aggregation regulator or modulator” refers to any agent that can regulate or modulate the aggregation of tau protein, that is, any agent that can either induce, promote or increase tau protein aggregation, or that can inhibit, prevent or reduce tau protein aggregation. Non-limiting example of tau protein aggregation regulator or modulator can include nucleic acids, proteins or metabolic factors.

Cells comprising one or more polynucleotides, expression cassettes, and/or vectors as described herein can be contacted with one or more putative tau protein aggregation regulators or modulators. The cells can be exposed to an excitation light. An emission light signal can be detected. Tau protein aggregation, or lack thereof can be detected in the cells; and tau protein aggregation regulator or modulator can be identified.

The method described herein can comprise culturing sensor cells described herein, and plating sensor cells at a cell density in a cell culture plate or dish.

Sensor cells can be contacted with a putative tau protein aggregation regulator or modulator, in the presence of in the absence of a cationic lipid reagent, such as Lipofectamine 2000 transfection reagent, for example.

Sensor cells can be separated from a cell culture dish or cell culture well and fixed prior to measuring a level of light signal emitted by a sensor cell, which can be correlated with the presence of tau protein aggregate in the cell, and therefore with the identification of a putative tau protein aggregation regulator or modulator.

Sensor cells comprising one or more polynucleotides, expression cassettes, vectors described herein can express a seed tau protein linked or fused to a donor fluorescent protein and a seed tau protein linked or fused to an acceptor fluorescent protein. The seed tau proteins can aggregate with one another to form tau protein aggregates, which can bring a donor fluorescent protein in close proximity to an acceptor fluorescent protein. Upon exposition of such cell to an excitation light, energy from a donor fluorescent protein can be transferred to an acceptor fluorescent protein, which can emit emission light signal that can be detected.

An emission light signal measured in a reporter cell in the presence of a sample known for not containing any tau protein can be used as a negative control. In such case, nothing disturbs the interaction between a seed tau protein linked to a donor fluorescent protein and a seed tau protein linked to an acceptor fluorescent protein; and an emission light signal can be detected. An emission light signal measured in a reporter cell in the presence of excessive amounts of exogenous tau protein can be used as a positive control. In such case, the interaction between a seed tau protein linked to a donor fluorescent protein and a seed tau protein linked to an acceptor fluorescent protein can be disturbed; and a lesser emission light signal (or no emission light signal) can be detected.

An emission light signal can be compared to a standard curve comparing emission light signals obtained in the presence of predetermined amounts of tau protein. For example, emission light signals measured in the presence of various concentrations of tau proteins or fragments can be used to generate a standard curve. If an emission light signal measured in a sample is less than a positive control, but greater than a negative control, it can then be compared to the emission light signal in a standard curve, to estimate the concentration (i.e., the titer) of tau proteins, fragments, or aggregates present in the sample.

Detecting more tau protein aggregates in the presence of a putative tau protein aggregation regulator or modulator can indicate that the putative tau protein aggregation regulator or modulator induces or promotes tau protein aggregation.

Detecting less tau protein aggregates in the presence of a putative tau protein aggregation regulator or modulator can indicate that the putative tau protein aggregation regulator or modulator inhibits or prevents tau protein aggregation.

The compositions and methods are more particularly described below, and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined below to provide additional guidance to the practitioner regarding the description of the compositions and methods. As used in the description herein and throughout the claims that follow, the meaning of “a”, “an”, and “the” includes plural reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, optional features, modification and variation of the concepts herein disclosed may be resorted to by those skilled in the art, and that such modifications and variations are considered to be within the scope of this invention as defined by the description and the appended claims.

Any single term, single element, single phrase, group of terms, group of phrases, or group of elements described herein can be each be specifically excluded from the claims.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods

In addition, where features or aspects of the invention are described in terms of Markush groups or other grouping of alternatives, those skilled in the art will recognize that the invention is also thereby described in terms of any individual member or subgroup of members of the Markush group or other group.

The following are provided for exemplification purposes only and are not intended to limit the scope of the invention described in broad terms above.

### EXAMPLES

### Example 1. Material and Methods

Generation of Biosensor Cell Lines (Tau RD(P301S)v2L and Tau RD(P301S)v2H).

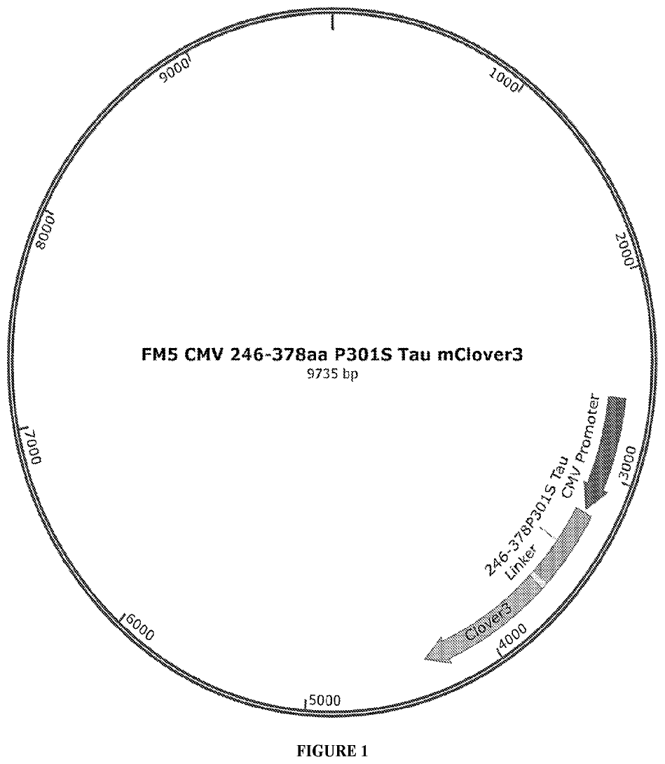

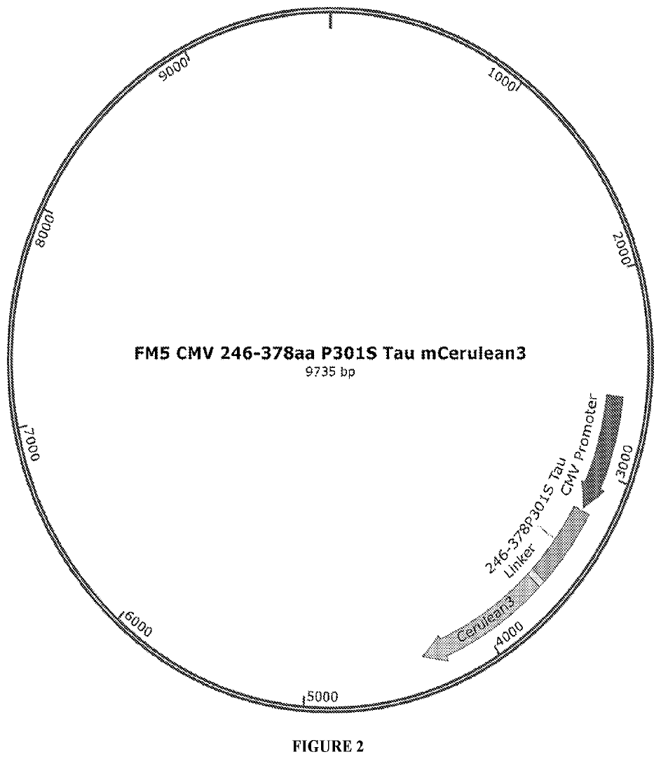

A lentiviral FM5-YFP plasmid containing the tau segment 246 to 378 with the P301S mutation, was used as a template, where the human ubiquitin C (Ubc) promoter was replaced with a human cytomegalovirus (CMV) promoter, and the YFP sequence was replaced with a mCerulean 3 or mClover3 coding sequences (see FIGS. 1 and 2). The sequence linking the tau segment and the coding sequence of the fluorophore (Cer or Co) was optimized to the following sequence: GSAGSAAGSGEF (SEQ ID NO:41). Low passage HEK293T cells (P5) were thawed and passaged with antibiotic free media twice before being co-administration of lentivirus encoding tau RD(P301S)-Clo/Cer tau lentivirus. After four passages, single cells were cell sorted by FACS based on low or high signal for both mCerulean3 and mClover3, termed version 2 low (v2L) or version 2 high (v2H). Monoclonal colonies were expanded and characterized as described.

Western Blot

Cell lysates were prepared by resuspending frozen cell pellets (˜1 million cells) in 100 uL of 0.25% Triton X-100 with protease inhibitors and incubating for 30 minutes on ice followed by centrifugation at 21,000×G for 15 minutes. Clarified supernatants were adjusted to a concentration of 1 mg/mL as determined by Pierce 660 nm assay and SDS-PAGE was performed with 5 ug of total protein loaded onto a 4-20% BisTris gel. After transferring the protein to a PVDF membrane, it was blocked with 5% milk in 0.1% TBS-T for 1 hr at room temperature. To detect tau protein HJ10.3, a mouse monoclonal antibody that binds the RD of tau, was used at a 1:10,000 dilution in blocking buffer for 4 hrs at RT. To detect the fluorescent proteins fused to tau, the Rockland anti-GFP antibody (cat. 600-101-215), which binds all GFP variants used in this work, was used at a 1:10,000 dilution, in blocking buffer. After blotting with appropriate secondary antibodies and imaging, the membranes were stripped and reblotted for GAPDH with (6C5, Fisher cat.NC9537307) at a 1:5000 dilution in blocking buffer.

Recombinant Tau Fibrils

Wild-type full-length (2N4R) tau was synthesized and purified. 8 μM purified recombinant tau was incubated with 8 uM heparin and 10 mM DTT at 37 C for 48 h in 10 mM HEPES, 100 mM NaCL, PH 7.4. The quality of fibrils was verified by transmission electron microscopy.

Human AD and Mouse Brain Tissue

All mice were housed and cared for according to the UT Southwestern animal care and use guidelines. Mice: Wild type C57BL/6 (stock #00064, Jackson Laboratory), Tau knockout (stock #007251), and PS19 mice expressing human 1N4R tau with the P301S mutation under control of the mouse prion promoter (Prnp)32 (stock #008169) mice, all 9 months old. All mice were transcardially perfused and the brains were removed and immediately flash froze. Frozen frontal cortex human brain tissues were obtained from 5 cases with a histopathological diagnosis of Alzheimer's disease from the brain bank of the Alzheimer's Disease Center UT Southwestern. Brain tissue was homogenized in 10% w/vol of 1×TBS with protease inhibitor cocktail (Roche) at 4° C. using a dounce homogenizer followed by intermittent probe sonication (Omni International) for 10 minutes. Homogenates were centrifuged at 21,000×g for 15 minutes at 4° C. to remove cellular debris and determined protein concentrations by BCA assay (Thermo Fisher).

Human CSF

Human lumbar CSF were obtained from the UT Southwestern O'Donnell Brain Institute Biorepository, along with clinical data including age, sex, and CSF t-tau, p-tau, and Aβ42 levels as measured by ADmark clinical assay (Athena Diagnostics).

Immunoprecipitation from CSF

50 μl of Dynabeads Protein A (Thermo Fisher) were washed per the manufacturer protocol and incubated with 10 μg of polyclonal antibody (TauA, Diamond Lab) against the first microtubule-binding repeat of tau for 1 hour at room temperature. washed beads were then added to 1 or 5 ml of human CSF and incubated with rotation overnight at 4° C. Captured proteins were eluted in low pH elution buffer (Pierce) and neutralized the buffer with 1:10 1M Tris pH 8.5 with a final volume of 120 μl.

Seeding Assays

P301S4 or P301S v2L or v2H HEK biosensor cells were plated in 96-well plates at 20,000 cells per well 24 hours before treatment. dilutions of recombinant tau fibrils or brain homogenates in Opti-MEM (Thermo Fisher), 30 μl total volume, or immunoprecipitation eluents 120 μl total volume, were allowed to come to room temperature. 1.5 μl of Lipofectamine 2000 transfection reagent (Invitrogen) was mixed with 28.5 μl of Opti-MEM for each sample and incubated at room temperature for 5 minutes before being mixing with the sample. After incubating the mixtures at room temperature for 30 minutes, they were divided among 3 wells of a 96-well plate. After 48 hours, cells were trypsinized and fixed in 2% PFA and suspended in flow cytometry buffer (1×HBSS, 1% FBS, 1 mM EDTA). percent FRET positivity was determined of each well by flow cytometry.

### Example 2. Results

Construction of Tau RD(P301S) v2H Biosensor Cells.

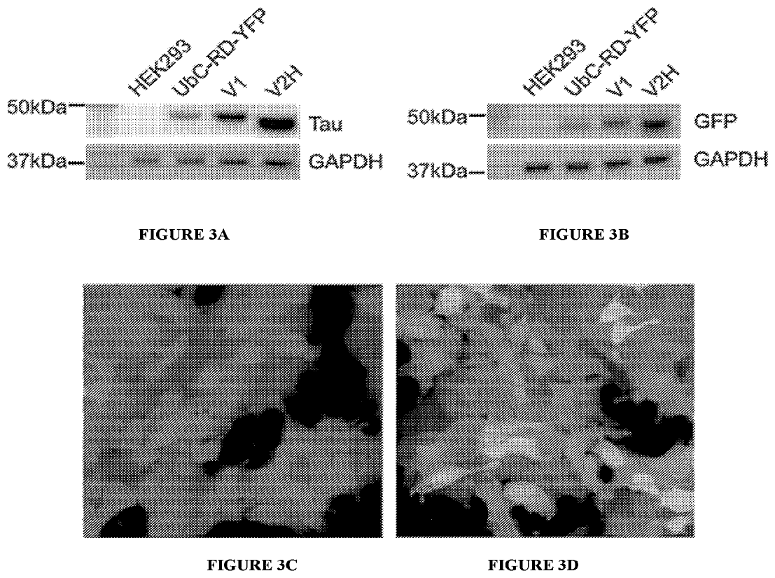

Upon sequencing of the plasmid used to express tau RD (P301S)-CFP/YFP, the Kozak sequence was modified to increase translation efficiency. In addition, the human ubiquitin (hUBC) promoter was replaced with the cytomegalovirus (CMV) promoter, and CFP and YFP were exchanged for brighter variants mClover3 (Clo) and mCerulean3 (Cer). The new constructs were cloned into a lentiviral expression vector, selected single colonies for characterization, and two clones with low and high tau expression that had minimal background FRET and induced strongly in response to exogenous seeds were picked. The version 2 low-expressing clone (v2L) was easier to grow, and reliably produced FRET upon exposure to tau seeds. The version 2 high-expressing clone (v2H) expressed higher levels of tau, and was more sensitive, but slightly more difficult to maintain in culture. v2L and v2H tau biosensors each expressed higher levels of intact tau RD-Clo/Cer fusions than the original biosensor line, as detected by western blots against tau-RD and GFP, and by fluorescence microscopy (FIGS. 3A-3D). The cells will be deposited at ATCC as Tau RD(P301S)v2L and Tau RD(P301S)v2H biosensors.

Increased Biosensor Sensitivity

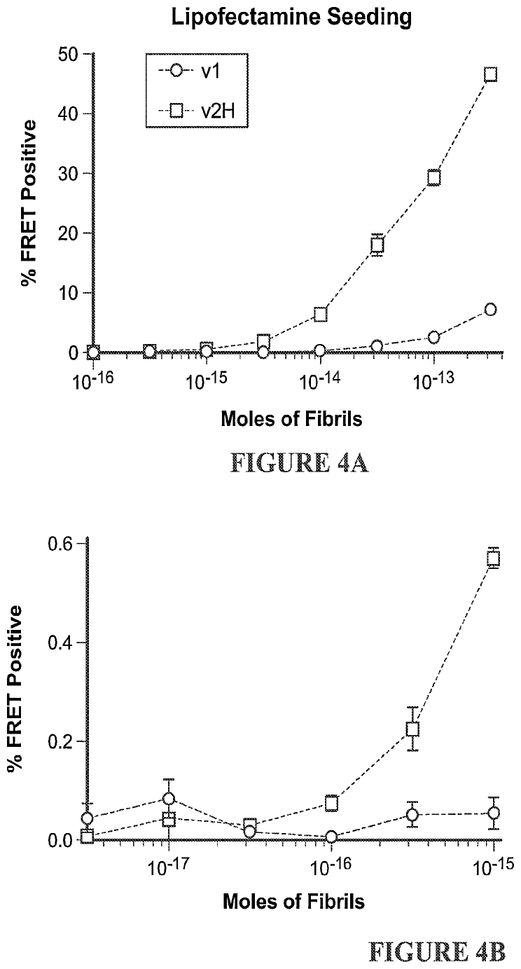

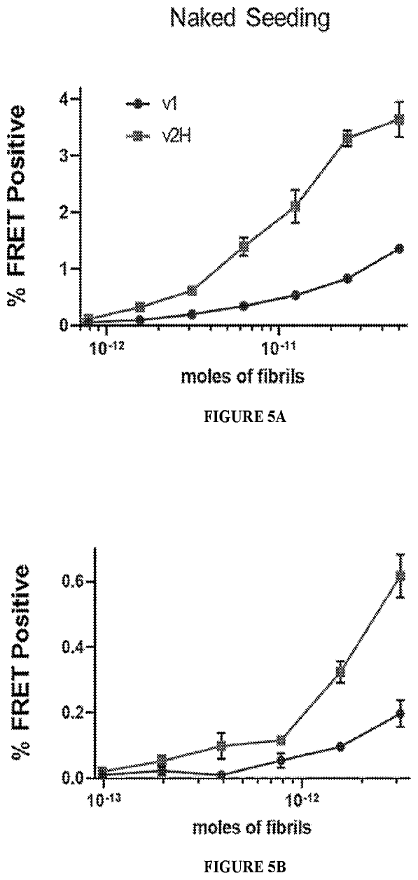

The sensitivity of v2L and v2H cells was first compared to the original line. synthetic fibrils based on exposure of recombinant tau protein to heparin were created. The fibrils were incubated with cells in the absence or presence of Lipofectamine 2000 for 48 h and quantified the percentage of FRET-positive cells using flow cytometry to determine the lower limit of detection. We detected 10 fM monomer equivalent in v2L cells and 32 aM in v2H cells (FIGS. 4A and 4B). v2L lines were approximately 10-fold more sensitive than the original line, the v2H line was more than 300-fold more sensitive than the original biosensor cell line. In the absence of cationic lipid reagent, 1.6 pM tau were detected in v2L cells and 0.78 pM tau in v2H cells (FIGS. 5A and 5B). The lower limit of detection (LLD), defined as the lowest quantity of tau fibrils that produces a signal of FRET positivity statistically distinguishable from background, was 10 fmol and 32 amol per sample for v1 and v2H respectively (Lipofectamine seeding) and 1.6 pmol and 0.78 pmol for v1 and v2H respectively (naked seeding). T-test, p<0.05.

Detection of Brain-Derived Tau Seeds

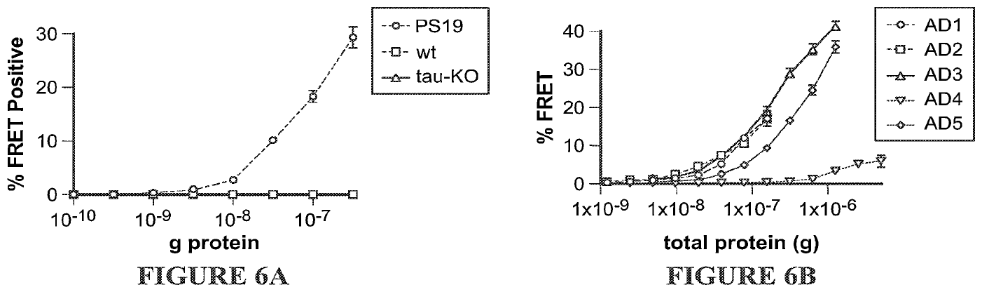

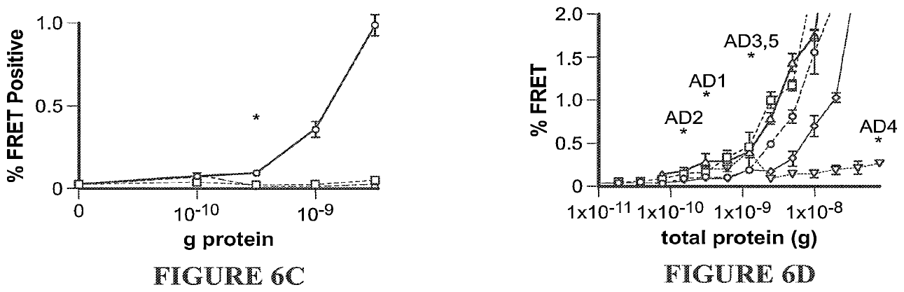

To evaluate detection mouse brain-derived tau, brain extract (10% w/v) from a 9-month-old PS19 transgenic mouse, which expresses full-length (1 N4R) human tau containing the P301S mutation were serially diluted. v2H cells were transduced using Lipofectamine 2000 (FIGS. 6A and 6C), and seeding activity was measured over four log orders of concentration. The lower limit of detection for lysate was ˜316 pg of total protein. Next frontal cortex homogenates from 5 AD cases were evaluated. The lower limit of detection ranged from 153 pg to 1.2 ng of total protein (FIGS. 6B and 6D).

The lower limit of detection ranged from 153 pg to 1.2 ng of total protein. Tau seeds can be efficiently purified from CSF. FRET positivity resulting from IP followed by seeding assay of spiked samples did not differ between CSF and PBS or with volume of IP. The LLD in this condition was 31.6 pg of total protein.

Efficient Purification and Detection of Tau Seeds from CSF

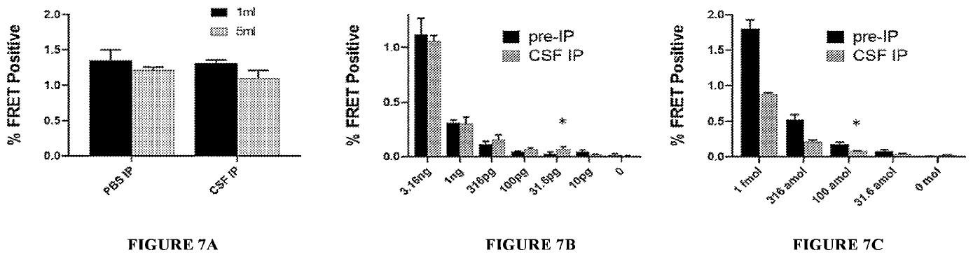

To determine the lower limit of detection of tau seeds in CSF, control CSF were spiked with small quantities of AD frontal cortex protein (brain AD1) or recombinant tau fibrils. the AD seeds were concentrated and purified using a rabbit polyclonal antibody directed against tau RD (TauA). (immunoprecipitation (IP) from either 1 or 5 ml of CSF vs. PBS recovered equivalent tau seeding activity (FIG. 7A) indicating that neither the volume of the IP, nor matrix effects from CSF proteins impact seed recovery. Next 1 ml aliquots of control CSF was spiked with successive dilutions of protein from AD frontal cortex or recombinant tau fibrils and immunopurification was performed, followed by the seeding assay. Seeding activity was detected in CSF from as little as 31.6 pg of total AD brain protein (FIG. 7B) and 100 attomolar (monomer equivalent) recombinant fibrils (FIG. 7C). As illustrated in FIG. 7, seeding was detected from spiked samples down to 31.6 pg of total AD brain protein and 100 attomoles tau monomer equivalent of recombinant fibrils. Pre-IP showed FRET positivity from direct treatment with the amount of protein spiked into the corresponding sample.

### Example 3. Discussion

Tau assemblies that act as templates for their own amplification (seeds) may underlie progression of neurodegenerative tauopathies, and assays that measure the levels of these pathogenic forms thus have great utility. While highly sensitive and specific conformational antibodies would be ideal, amplification of tau seeds in purified systems (REF) or in cultured “biosensor” cells can be used for sensitive and specific detection of pathological tau. Reliable detection of tau seeding activity in a peripheral fluid such as CSF could be transformational in disease characterization.

Consequently, it was tried repeatedly to detect pathological tau in human CSF or blood. Herein, expression of tau RD(P301S)-Clo/Cer was optimized in HEK293T cells and a biosensor with >300-fold improved sensitivity versus the original line was created. The increase in sensitivity was especially notable for cationic lipid-enhanced delivery. The v2H line should be especially useful to quantify tau seeds that are of low abundance. Given their ease of culture, the v2L line may be more useful to quantify seeding in samples with stronger signal. Given the demonstrated utility of the original biosensor assays to detect early evidence of tau pathology in brain tissue, it is anticipated that the v2H cell line will enhance detection of pathological tau beyond current capability.

The source of tau in the CSF in AD is unclear, but is unlikely to be due to cell death, because not all tauopathies exhibit progressive increases in CSF tau. Stable isotope labeling kinetic studies of tau metabolism and turnover in human neurons have found a regulated truncation and secretion of tau species containing only N-terminal and mid-regions. While total tau levels in the CSF can rise due to passive release with neuronal death, such as in acute stroke, elevated CSF tau in AD patients represents truncated, rather than full-length species, indicating that it is likely driven by differences in processing and secretion. Seed-competent tau can be released into the extracellular fluid in cell culture models. It is not clear whether this occurs in the brains of AD patients, but it is promising that ultra-sensitive RT-QuIC in vitro assays have demonstrated tau seeding in post-mortem CSF, though at many orders of magnitude lower levels than in brain. Post-mortem CSF may contain intracellular tau released after death, and thus pre-mortem CSF is a more accurate reflection of clinical utility. A 4R RT-QuIC assay sensitive for PSP and CBD seeds showed higher mean signal in groups of PSP and CBD pre-mortem CSF relative to a group of controls.

What is claimed is:

1. A polynucleotide comprising:
(a) a polynucleotide encoding a tau repeat domain comprising SEQ ID NO:1; and
(b) a polynucleotide encoding a reporter.

2. The polynucleotide of claim 1, further comprising:
(a) a polynucleotide encoding a promoter; and
(b) a polynucleotide encoding a linker.

3. The polynucleotide of claim 1, wherein the polynucleotide encoding a reporter comprises SEQ ID NO:2 or SEQ ID NO:3.

4. The polynucleotide of claim 2, wherein the polynucleotide encoding a promoter comprises SEQ ID NO:4.

5. The polynucleotide of claim 2, wherein the polynucleotide encoding a linker comprises SEQ ID NO:5.

6. A vector comprising an expression cassette comprising the polynucleotide of claim 1.

7. The vector of claim 6, comprising SEQ ID NO:6 or 7.

8. A cell comprising:
(i) a first vector comprising a polynucleotide encoding a tau repeat domain and a first reporter, and a second vector comprising a polynucleotide encoding a tau repeat domain and a second reporter; or
(ii) a vector comprising a first polynucleotide encoding a tau repeat domain and a first reporter, and a second polynucleotide encoding a tau repeat domain and a second reporter.

9. The cell of claim 8, wherein the first reporter comprises SEQ ID NO:2 or SEQ ID NO:3.

10. The cell of claim 8, wherein the first polynucleotide comprises SEQ ID NO:1 and SEQ ID NO:2.

11. The cell of claim 8, wherein the second polynucleotide comprises SEQ ID NO:1 and SEQ ID NO:3.

12. The cell of claim 8, wherein the first polynucleotide comprises SEQ ID NO:6, and wherein the second polynucleotide comprises SEQ ID NO:7.

13. The cell of claim 12, expressing Tau RD(P301S).

14. A method of measuring a titer of or of detecting a seed tau protein in a sample comprising:
(a) contacting the sample with the cell of claim 8;
(b) performing a seeding assay; and
(c) detecting tau protein aggregates,
thereby measuring a titer of or of detecting seed tau protein in the sample.

15. A method of detecting Alzheimer's disease (AD), or a neurodegenerative tauopathy disease or condition linked to tau protein aggregation in a subject comprising:
(a) contacting a sample with the cell of claim 8;
(b) performing a seeding assay; and
(c) detecting tau protein aggregates,
thereby detecting AD or neurodegenerative tauopathy disease or condition in a subject.

16. The method of claim 15, wherein the sample is a biological fluid, a tissue sample, a cerebrospinal fluid, a brain homogenate, or an aggregated material amplified in vitro therefrom.

17. The method of claim 15, wherein tau protein present in the sample is immunoprecipitated prior to performing step (a).

18. The method of claim 15, wherein the method detects about as low as 10 pg/ml of tau protein in the sample.

19. A method of identifying a tau protein aggregation inhibitor comprising:
(a) contacting the cell of claim 8 with a putative tau protein aggregation inhibitor;
(b) performing a seeding assay;
(c) detecting tau protein aggregates, and
(d) identifying a tau protein aggregation inhibitor,
wherein a tau protein aggregation inhibitor interacts with tau protein.

20. The method of claim 19, wherein detecting tau protein aggregates indicates that the putative tau protein aggregation inhibitor does not inhibit tau protein aggregation.

21. The method of claim 19, wherein a lack of detection of tau protein aggregates indicates that the putative tau protein aggregation inhibitor inhibits tau protein aggregation.

22. A method of detecting attomolar levels of a seed tau protein in a sample comprising:
(a) contacting the sample with the cell of claim 8;
(b) performing a seeding assay; and
(c) detecting tau protein aggregates,
thereby detecting seed tau protein present at attomolar levels in the sample.

23. A method of identifying tau protein aggregation regulator or modulator comprising:
(a) contacting the cell of claim 8 with a putative tau protein aggregation regulator or modulator;
(b) performing a seeding assay; and
(c) detecting change in tau protein aggregation in the cell,
thereby identifying putative tau protein aggregation regulator or modulator.

24. The method of claim 23, wherein the tau protein aggregation regulator or modulator is a nucleic acid, a protein or a metabolic factor.

25. The method of claim 23, wherein detecting more tau protein aggregates in the presence of a putative tau protein aggregation regulator or modulator indicates that the putative tau protein aggregation regulator or modulator induces or promotes tau protein aggregation.

26. The method of claim 23, wherein detecting less tau protein aggregates in the presence of a putative tau protein aggregation regulator or modulator indicates that the putative tau protein aggregation regulator or modulator inhibits or prevents tau protein aggregation.
